(19) United States
(12) Patent Application Publication
(10) Pub. No.: US 2025/0064760 A1
(43) Pub. Date: Feb. 27, 2025

(54) INTRACANALICULAR INSERT COMPRISING A NON-STEROIDAL ANTI-INFLAMMATORY AGENT

(71) Applicant: OCULAR THERAPEUTIX, INC., Bedford, MA (US)

(72) Inventors: Charles D. BLIZZARD, Bedford, MA (US); Ankita DESAI, Bedford, MA (US); Rami EL-HAYEK, Bedford, MA (US); Michael GOLDSTEIN, Bedford, MA (US); Peter JARRETT, Bedford, MA (US); Oktay UZUN, Bedford, MA (US)

(21) Appl. No.: 18/726,302

(22) PCT Filed: Jan. 6, 2023
(86) PCT No.: PCT/US2023/010319

§ 371 Date: Jul. 2, 2024

Related U.S. Application Data
(60) Provisional application No. 63/297,776, filed on Jan. 9, 2022.

(51) Int. Cl. A61K 31/165; A61K 9/00

(52) U.S. Cl. CPC A61K 31/165; A61K 9/0051

(57) ABSTRACT

In certain embodiments, the invention relates to a sustained release biodegradable intracanalicular insert containing nepafenac dispersed in a hydrogel for the treatment of pain and inflammation. According to certain embodiments of the present invention, pain and inflammation is treated by administering a biodegradable insert into the superior and/or inferior canaliculus of the eye, wherein the insert provides sustained release of a nonsteroidal anti-inflammatory such as nepafenac that is effective for treating pain and inflammation in a patient, e.g., during a period of one or more weeks, with only a single administration, and without the need for removal of the drug-depleted insert.

### TECHNICAL FIELD

The present invention relates to the treatment of pain and inflammation. According to certain embodiments of the present invention, pain and inflammation is treated by administering a biodegradable insert into the superior and/or inferior canaliculus of the eye, wherein the insert provides sustained release of nepafenac.

### BACKGROUND

Pain and inflammation is a frequently encountered ocular condition. It typically presents itself after cataract surgery among other clinical manifestations. Pain and inflammation is commonly treated with nonsteroidal anti-inflammatory eye drops. A specific issue with currently available eye drop formulations. Ophthalmic drops may have to be administered several times per day as a large portion of the active ingredient is washed out quickly out of the eye and therefore exposure of the eye surface to the active agent may be short. For this reason, formulations often maximize concentration to compensate for this inefficiency, which may be associated with acute high concentrations on the ocular surface that may result in safety issues. In addition, burning, itching and stinging associated with preservatives, such as anti-microbial preservatives, included in ophthalmic drops may be observed. Further, as patients may need to administer the drops multiple times per day, daily life is highly affected and patient compliance may be low. As the administration of drops into the eye can be perceived as difficult, accuracy of drop delivery to the ocular surface may also be limited. Over- or underdosing may thus occur.

In view of the drawbacks and challenges experienced with current available treatments, novel treatment methods, which effectively deliver nonsteroidal anti-inflammatory agents in an appropriate dose and are effective over an extended period to treat pain and inflammation of one or more weeks, while avoiding the need for daily nonsteroidal anti-inflammatory administrations would provide benefits for patients.

### OBJECTS AND SUMMARY OF THE INVENTION

It is an object of certain embodiments of the present invention to provide an ocular insert comprising a nonsteroidal anti-inflammatory such as nepafenac that is effective for treating pain and inflammation in a patient, e.g., during a period of one or more weeks.

Another object of certain embodiments of the present invention is to provide an ocular insert comprising a nonsteroidal anti-inflammatory such as nepafenac that provides for sustained release of the nonsteroidal anti-inflammatory and/or metabolite to the ocular surface through the tear fluid.

Another object of certain embodiments of the present invention is to provide an ocular insert comprising nepafenac that provides for a therapeutically effective amount of amfenac in the aqueous humor, e.g., after 3 days, after 5 days, after 7 days, after 14 days, after 21 days or after 30 days post administration.

Another object of certain embodiments of the present invention is to provide an ocular insert comprising a nonsteroidal anti-inflammatory such as nepafenac that provides for sustained release of the nonsteroidal anti-inflammatory and/or metabolite to the ocular surface through the tear fluid, wherein the period of sustained release comprises a period of constant or substantially constant nonsteroidal anti-inflammatory release per day.

Another object of certain embodiments of the present invention is to provide an ocular insert comprising a nonsteroidal anti-inflammatory such as nepafenac that provides for a treatment of pain and inflammation for a period of one or more weeks, with only a single administration.

Another object of certain embodiments of the present invention is to provide an ocular insert comprising a nonsteroidal anti-inflammatory such as nepafenac that is sufficiently biodegradable, thereby avoiding the need for removal of the drug-depleted insert.

Another object of certain embodiments of the present invention is to provide an ocular insert comprising a nonsteroidal anti-inflammatory such as nepafenac that is biocompatible and low or non-immunogenic due to certain embodiments of the insert being free of animal- or human-derived components.

Another object of certain embodiments of the present invention is to provide an ocular insert comprising a nonsteroidal anti-inflammatory such as nepafenac that is free of anti-microbial preservatives.

Another object of certain embodiments of the present invention is to provide an ocular insert comprising a nonsteroidal anti-inflammatory such as nepafenac that is dimensionally stable when in a dry state but changes its dimensions upon hydration, e.g., after administration to the eye.

Another object of certain embodiments of the present invention is to provide an ocular insert comprising a nonsteroidal anti-inflammatory such as nepafenac that is administered in a dry state and hydrates when inserted e.g., into the canaliculus.

Another object of certain embodiments of the present invention is to provide an ocular insert comprising a nonsteroidal anti-inflammatory such as nepafenac that in its dry state is easy to administer but that is firmly secured in the canaliculus, avoiding potential insert loss during the treatment period, thereby providing improved retention, especially when compared to commonly applied plugs such as collagen or silicone plugs.

Another object of certain embodiments of the present invention is to provide an ocular insert comprising a nonsteroidal anti-inflammatory such as nepafenac, wherein the insert is stable and has a defined shape and surface area both prior to as well as after insertion (i.e., inside the canaliculus).

Another object of certain embodiments of the present invention is to provide an ocular insert comprising a nonsteroidal anti-inflammatory such as nepafenac that is easy to handle, in particular that does not spill or fragment easily.

Another object of certain embodiments of the present invention is to provide an ocular insert comprising a nonsteroidal anti-inflammatory such as nepafenac that enables administration of an exact dose (within a broad dose range), thereby avoiding the risk of over- and under-dosing.

Another object of certain embodiments of the present invention is to provide an ocular insert comprising a nonsteroidal anti-inflammatory such as nepafenac that does not cause nonsteroidal anti-inflammatory peaks or substantial peaks that could potentially result in adverse effects.

Another object of certain embodiments of the present invention is to provide an ocular insert comprising a nonsteroidal anti-inflammatory such as nepafenac for the treatment of pain and inflammation such as the acute or chronic treatment of pain and inflammation that provides a lower incidence of side effects, such as burning, stinging or itching, as compared to commonly known pain and inflammation therapies.

Another object of certain embodiments of the present invention is to provide an ocular insert comprising a nonsteroidal anti-inflammatory such as nepafenac that provides a hands-free alternative for the patient compared to conventional pain and inflammation treatments.

Another object of certain embodiments of the present invention is to provide an ocular insert comprising a nonsteroidal anti-inflammatory such as nepafenac that generally stays in the area of the eye to which it was administered, such as in the inferior and/or superior (vertical) canaliculus.

Another object of certain embodiments of the present invention is to provide an ocular insert comprising a nonsteroidal anti-inflammatory such as nepafenac that has increased patient compliance as compared to currently available pain and inflammation treatments.

Another object of certain embodiments of the present invention is to provide an ocular insert comprising a nonsteroidal anti-inflammatory such as nepafenac that can be visualized in a fast and simple manner and by a non-invasive method.

Another object of certain embodiments of the present invention is to provide an ocular insert comprising a nonsteroidal anti-inflammatory such as nepafenac that provides for sustained release of a therapeutically effective amount of the nonsteroidal anti-inflammatory such as nepafenac and/or amfenac over an extended period of time, such as over a period of up to about 7 days, up to about 14 days, or up to about 21 days, or up to about 30 days or up to 42 days after administration.

Another object of certain embodiments of the present invention is to provide an ocular insert comprising a nonsteroidal anti-inflammatory such as nepafenac that releases a constant or essentially constant amount of the nonsteroidal anti-inflammatory such as nepafenac and/or metabolite over an extended period of time, such as for a period of up to about 7 days, or up to about 14 days, or up to about 21 days, or up to about 30 days or up to about 42 days after administration.

Another object of certain embodiments of the present invention is to provide an ocular insert comprising a nonsteroidal anti-inflammatory such as nepafenac that provides for sustained release of a therapeutically effective amount of the nonsteroidal anti-inflammatory such as nepafenac and/or metabolite over an extended period of time after administration, such as over a period of up to about 7 days, up to about 14 days, or up to about 21 days, or up to about 30 days or up to about 42 days, thereby avoiding the need for frequent nonsteroidal anti-inflammatory administrations, which are required e.g. several times a day when using ophthalmic drops.

Another object of certain embodiments of the present invention is to provide an ocular insert comprising a nonsteroidal anti-inflammatory such as nepafenac that provides for sustained release of a therapeutically effective amount of the nonsteroidal anti-inflammatory such as nepafenac or metabolite over an extended period of time after administration, such as for a period of up to about 7 days, up to about 14 days, or up to about 21 days, or up to about 30 days or up to about 42 days, wherein the nonsteroidal anti-inflammatory amount in the tear film is consistently maintained at a therapeutically effective level sufficient for anti-inflammatory therapy of the ocular surface.

Another object of certain embodiments of the present invention is to provide an ocular insert comprising a nonsteroidal anti-inflammatory such as nepafenac that provides for sustained release of a therapeutically effective amount of the nonsteroidal anti-inflammatory such as nepafenac and/or metabolite over an extended period of time after administration, such as for a period of up to about 7 days, up to about 14 days, or up to about 21 days, or up to about 30 days or up to about 42 days, wherein essentially no toxic concentrations of the nonsteroidal anti-inflammatory are observed on the ocular surface and/or in the tear film.

Another object of certain embodiments of the present invention is to provide an ocular insert comprising a nonsteroidal anti-inflammatory such as nepafenac that provides for sustained release of a therapeutically effective amount of the nonsteroidal anti-inflammatory such as nepafenac and/or metabolite over an extended period of time after administration, such as for a period of up to about 7 days, up to about 14 days, or up to about 21 days, or up to about 30 days or up to about 42 days, wherein the nonsteroidal anti-inflammatory is not resorbed systemically or not substantially resorbed systemically thereby minimizing or avoiding systemic toxicity.

Another object of certain embodiments of the present invention is to provide a method of treating pain and inflammation in a patient in need thereof with an ocular insert as disclosed herein.

Another object of certain embodiments of the present invention is to provide a method of manufacturing an ocular insert comprising a nonsteroidal anti-inflammatory such as nepafenac.

One or more of these objects of the present invention and others are solved by one or more embodiments of the invention as disclosed and claimed herein.

### BRIEF DESCRIPTION OF THE DRAWINGS

FIG. 1 Schematic representation of an exemplary insert packaging. The insert is placed into a foam carrier and sealed with a foil pouch.

FIG. 2 Schematic exemplary representation of insert placement into the inferior vertical canaliculus through the lower punctum of the eye (A). Visualization of the insert is possible e.g., by illumination with blue light (B). The fluorescein in the intracanalicular insert in one embodiment illuminates when excited with blue light enabling confirmation of insert presence in a non-invasive manner.

FIG. 3 depicts the tear fluid concentrations of nepafenac and amfenac in tear fluid in beagles.

FIG. 4 depicts results of Example 3.

FIG. 5 depicts results of Example 4.

FIG. 6 depicts results of Example 4.

FIG. 7 depicts results of Example 4.

FIG. 8 depicts results of Example 5.

### DEFINITIONS

The term “insert” as used herein refers to an object that contains an active agent, specifically a nonsteroidal anti-inflammatory such as nepafenac and that is administered into the human or animal body, such as to the canaliculus of the eye (one eye or both eyes, as well as inferior and/or superior canaliculus), where it remains for a certain period of time while it releases the active agent into the surrounding environment. An insert can have any predetermined shape before being inserted, which general shape may be maintained to a certain degree upon placing the insert into the desired location, although dimensions of the insert (e.g., length and/or diameter) may change after administration due to hydration as further disclosed herein. In other words, what is administered into the canaliculus of the eye is not a solution or suspension, but an already shaped, coherent object. The insert has thus been completely formed, e.g., according to the methods disclosed herein prior to being administered. Over the course of time the inserted insert in certain embodiments is biodegraded (as disclosed herein), and may thereby change its shape (e.g., may expand in diameter and decrease in length) until it has been completely dissolved/resorbed. Herein, the term “insert” is used to refer both to an insert in a hydrated (also referred to herein as “wet”) state when it contains water, e.g., after the insert has been hydrated or re-hydrated once administered to the eye or otherwise immersed into an aqueous environment (such as in vitro), as well as to an insert in its/a dry (dried/dehydrated) state. Thus, in certain embodiments, an insert in its dry/dried state in the context of the present invention may contain no more than about 1% by weight water. The water content of an insert in its dry/dried state may be measured e.g., by means of a Karl Fischer coulometric method. Whenever dimensions of an insert (i.e., length, diameter, or volume) are reported herein in the hydrated state, these dimensions are measured after the insert has been immersed in phosphate-buffered saline at a pH value of 7.4 at 37° C. for 24 hours. Whenever dimensions of an insert are reported herein in the dry state, these dimensions are measured after the insert has been fully dried (and thus, in certain embodiments, contains no more than about 1% by weight water). In certain embodiments, the insert is kept in an inert atmosphere glove box containing below 20 ppm of both oxygen and moisture for at least about 7 days.

In certain embodiments of the present invention, the term “fiber” (used interchangeably herein with the term “rod”) characterizes an object (i.e., in the present case an insert according to certain embodiments of the present invention) that in general has an elongated shape. Specific dimensions of inserts of the present invention are disclosed herein. The insert may have a cylindrical or essentially cylindrical shape, or may have a non-cylindrical shape. The cross-sectional area of the fiber or the insert may be either round or essentially round, but may in certain embodiments also be oval or oblong, or may in other embodiments have different geometries, such as cross-shaped, star-shaped or other as disclosed herein.

The term “ocular” as used herein refers to the eye in general, or any part or portion of the eye (as an “ocular insert” according to the invention refers to an insert that can in principle be administered to any part or portion of the eye). The present invention in certain embodiments is directed to intracanalicular administration of an ocular insert (in this case the “ocular insert” is thus an “intracanalicular insert”), and to the treatment of pain and inflammation.

The term “biodegradable” as used herein refers to a material or object (such as the intracanalicular insert according to the present invention) which becomes degraded in vivo, i.e., when placed in the human or animal body. In the context of the present invention, as disclosed in detail herein, the insert comprising the hydrogel within which particles of a nonsteroidal anti-inflammatory, such as particles of nepafenac, are dispersed, slowly biodegrades over time once deposited within the eye, e.g., within the canaliculus. In certain embodiments, biodegradation takes place at least in part via ester hydrolysis in the aqueous environment provided by the tear fluid. In certain embodiments, the intracanalicular inserts of the present invention slowly soften and liquefy over time. In certain embodiments, they are eventually cleared (disposed/washed out) through the nasolacrimal duct.

A “hydrogel” is a three-dimensional network of one or more hydrophilic natural or synthetic polymers (as disclosed herein) that can swell in water and hold an amount of water while maintaining or substantially maintaining its structure, e.g., due to chemical or physical cross-linking of individual polymer chains. Due to their high water content, hydrogels are soft and flexible, which makes them very similar to natural tissue. In the present invention the term “hydrogel” is used to refer both to a hydrogel in the hydrated state (also referred to herein synonymously as the “wet state”) when it contains water (e.g. after the hydrogel has been formed in an aqueous solution, or after the hydrogel has been hydrated or re-hydrated once inserted into the eye or otherwise immersed into an aqueous environment) and to a hydrogel in its/a dry (dried/dehydrated) state when it has been dried to a low water content of e.g. not more than 1% by weight as disclosed herein. In the present invention, wherein an active principle is contained (e.g., dispersed) in a hydrogel, the hydrogel may also be referred to as a “matrix”.

The term “polymer network” as used herein describes a structure formed of polymer chains (of the same or different molecular structure and of the same or different average molecular weight) that are cross-linked with each other. Types of polymers suitable for the purposes of the present invention are disclosed herein. The polymer network may be formed with the aid of a crosslinking agent as also disclosed herein.

The term “amorphous” refers to a polymer or polymer network or other chemical substance or entity which does not exhibit crystalline structures in X-ray or electron scattering experiments.

The term “semi-crystalline” refers to a polymer or polymer network or other chemical substance or entity which possesses some crystalline character, i.e., exhibits some crystalline properties in X-ray or electron scattering experiments.

The term “crystalline” refers to a polymer or polymer network or other chemical substance or entity which has crystalline character as evidenced by X-ray or electron scattering experiments. The term “precursor” or “polymer precursor” or specifically “PEG precursor” herein refers to those molecules or compounds that are reacted with each other and that are thus connected via crosslinks to form a polymer network and thus the hydrogel matrix. While other materials might be present in the hydrogel, such as active agents, visualization agents or buffers, they are not referred to as “precursors”.

The molecular weight of a polymer precursor as used for the purposes of the present invention and as disclosed herein may be determined by analytical methods known in the art. The molecular weight of polyethylene glycol can for example be determined by any method known in the art, including gel electrophoresis such as SDS-PAGE (sodium dodecyl sulphate-polyacrylamide gel electrophoresis), gel permeation chromatography (GPC), including GPC with dynamic light scattering (DLS), liquid chromatography (LC), as well as mass spectrometry such as matrix-assisted laser desorption/ionization-time of flight (MALDI-TOF) spectrometry or electrospray ionization (ESI) mass spectrometry. The molecular weight of a polymer, including a polyethylene glycol precursor as disclosed herein, is an average molecular weight (based on the polymer's molecular weight distribution), and may therefore be indicated by means of various average values, including the weight average molecular weight (Mw) and the number average molecular weight (Mn). Any of such average values may generally be used in the context of the present invention. In the context of the present invention, the average molecular weight of the polyethylene glycol units or other precursors or units as disclosed herein is the number average molecular weight (Mn) and is indicated in the unit “Daltons”.

The parts of the precursor molecules that are still present in a final polymer network are also called “units” herein. The “units” are thus the building blocks or constituents of a polymer network forming the hydrogel. For example, a polymer network suitable for use in the present invention may contain identical or different polyethylene glycol units as further disclosed herein.

As used herein, the term “crosslinking agent” or “crosslinker” refers to any molecule that is suitable for connecting precursors via crosslinks to form the polymer network and thus the hydrogel matrix. In certain embodiments, crosslinking agents may be low-molecular weight compounds or may be polymeric compounds as disclosed herein.

The term “sustained release” is generally defined for the purposes of the present invention to refer to pharmaceutical dosage forms or products (in the case of the present invention these products are inserts) which are formulated to make an active, such as a nonsteroidal anti-inflammatory according to the present invention, specifically including but not limited to nepafenac, available over an extended period of time after administration, such as one or more weeks, thereby allowing a reduction in dosing frequency compared to an immediate release dosage form, e.g. a solution of a nonsteroidal anti-inflammatory that is topically applied onto the eye (i.e. nonsteroidal anti-inflammatory-comprising eye drops). Other terms that may be used herein interchangeably with “sustained release” are “extended release” or “controlled release”. “Sustained release” thus generally characterizes the release of an API, specifically, the nonsteroidal anti-inflammatory, such as nepafenac, that is contained in an insert according to the present invention. The term “sustained release” per se is not associated with or limited to a particular rate of (in vitro or in vivo) release, although in certain embodiments of the invention an insert may be characterized by a certain average rate of (in vitro or in vivo) release or a certain release profile as disclosed herein. As an insert of the present invention (whether explicitly referred to herein as a “sustained release” insert or simply as an “insert”) provides for sustained release of the API, an insert of the present invention may therefore also be referred to as a “depot”.

Within the specific meaning of the present invention, the term “sustained release” also comprises a period of constant or substantially constant (i.e., above a certain level) nonsteroidal anti-inflammatory release per day when this period of constant or substantially constant release is followed by a period of tapered nonsteroidal anti-inflammatory release. In such specific case, an overall sustained release provided by an insert of the present invention (as defined above) may mean that the release rate is not necessarily constant or essentially constant throughout the entire period of nonsteroidal anti-inflammatory release, but may change over time as just described (i.e., with an initial period of constant or essentially constant, i.e., sustained release, followed by a period of tapered release). Within the meaning of the invention, the term “tapered” or “tapering” refers to a decreasing release of nonsteroidal anti-inflammatory such as nepafenac over time until the nonsteroidal anti-inflammatory is completely released.

The term “visualization agent” as used herein refers to a molecule or composition that may be contained within an insert of the present invention and that provides the possibility of easily visualizing the insert in a non-invasive manner when it is located in the canaliculus of the eye, e.g., by illuminating the corresponding eye parts with a suitable light source, such as blue light. The visualization agent may be a fluorophore such as fluorescein, rhodamine, coumarin, and cyanine, or other suitable agents as disclosed herein. In certain embodiments the visualization agent is fluorescein or includes a fluorescein moiety.

As used herein, the term “ocular surface” comprises the conjunctiva and the cornea, together with elements such as the lacrimal apparatus, including the lacrimal punctum, as well as the lacrimal canaliculus and associated eyelid structures. Within the meaning of this invention, the ocular surface encompasses also the aqueous humor.

As used herein, the terms “tear fluid” or “tears” or “tear film” refer to the liquid secreted by the lacrimal glands, which lubricates the eyes. Tears are made up of water, electrolytes, proteins, lipids, and mucins.

As used herein, the term “bilaterally” or “bilateral” refers (in the context of administration of the inserts of the present invention) to an administration of the inserts into both eyes of a patient. “Unilaterally” or “unilateral” thus refers to an administration of the insert into one eye only. The inserts may be independently inserted into the superior and/or the inferior canaliculus of both eyes or of one eye.

As used herein, the terms “administration” or “administering” or “administered” etc. in the context of the inserts of the present invention refer to the process of insertion of the inserts through the opening of the punctum into the canaliculus of the eye. Thus, “administering an insert” or similar terms refer to the insertion of the insert into the canaliculus. The terms “insertion” or “inserting” or “inserted” etc. in the context of the inserts of the present invention equally refer to the process of insertion of the inserts through the opening of the punctum into the canaliculus of the eye and are thus herein used interchangeably with the terms “administration” or “administering” or “administered”. In contrast, the terms “administration” or “administering” or “administered” etc. in the context of topical ophthalmic pharmacological products such as eye drops (which are not the subject of the present invention) refer to topical application of these products onto the eye.

As used herein, the term “insert stacking” or “stacking” refers to the insertion of a further insert on top of a first insert while the first insert is still retained in the canaliculus (because it has not yet sufficiently biodegraded and/or has not yet cleared through the nasolacrimal duct). In certain embodiments, the further insert is placed on top of the first insert after the nonsteroidal anti-inflammatory contained in the first insert is completely or essentially completely released, or after at least about 70% or at least about 80% or at least about 90% of the nonsteroidal anti-inflammatory contained in the first insert has been released. Insert stacking enables, for instance, prolonged nonsteroidal anti-inflammatory treatment.

The term “plug” as used herein refers to a device capable of providing an occlusion, substantial occlusion or partial occlusion of the tear duct(s) (“lacrimal occlusion”) thereby minimizing or preventing draining of tears. A plug thus increases tear retention, which helps to keep the eyes moist. Plugs can be classified into “punctal plugs” and “intracanalicular plugs”. Intracanalicular plugs are also referred to as “canalicular plugs” in literature. Both plug classes are inserted through the upper and/or lower punctum of the eye. Punctal plugs rest at the punctal opening making them easily visible and, hence, removable without much difficulty. However, punctal plugs may show poor retention rates and can be more easily contaminated with microbes due to their exposed localization which may result in infection. In contrast, intracanalicular plugs are essentially not visible and provide a better retention rate compared to punctal plugs as they are placed inside either the vertical or the horizontal canaliculus. However, currently available intracanalicular plugs may not be easy to remove and/or may provide an increased risk of migration due to loose fit. Commercially available plugs are often made of collagen, acrylic polymers, or silicone.

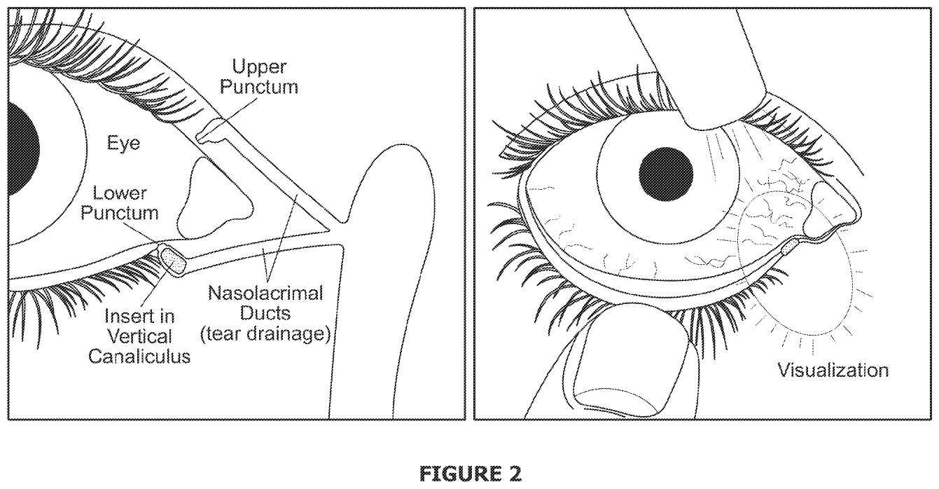

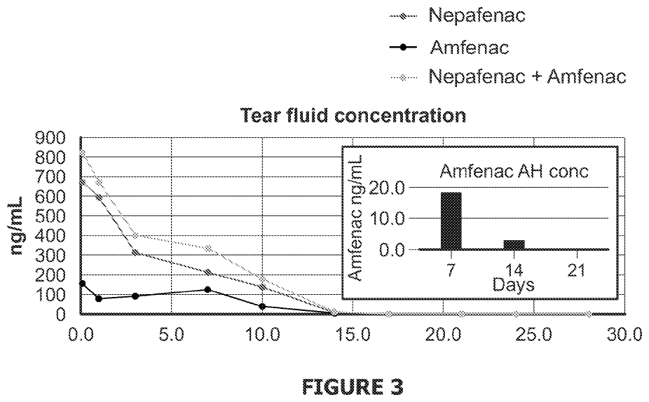

The terms “canaliculus” (plural “canaliculi”) or alternatively “tear duct” as used herein refer to the lacrimal canaliculus, i.e., the small channels in each eyelid that drain lacrimal fluid (tear fluid) from the lacrimal punctum to the nasolacrimal duct (see also FIG. 2). Canaliculi therefore form part of the lacrimal apparatus that drains lacrimal fluid from the ocular surface to the nasal cavity. The canaliculus in the upper eyelid is referred to as “superior canaliculus” or “upper canaliculus”, whereas the canaliculus in the lower eyelid is referred to as “inferior canaliculus” or “lower canaliculus”. Each canaliculus comprises a vertical region, referred to as “vertical canaliculus” following the lacrimal punctum and a horizontal region, referred to as “horizontal canaliculus” following the vertical canaliculus, wherein the horizontal canaliculus merges into the nasolacrimal duct.

The term “punctum” (plural “puncta”) refers to the lacrimal punctum, an opening on the margins of the eyelids, representing the entrance to the canaliculus. After tears are produced, some fluid evaporates between blinks, and some is drained through the lacrimal punctum. As both the upper and the lower eyelids show the lacrimal punctum, the puncta are therefore referred to as “upper punctum” or “superior punctum” and “lower punctum” or “inferior punctum”, respectively (see also FIG. 2).

The term “intracanalicular insert” refers to an insert that can be administered through the upper and/or lower punctum into the superior and/or inferior canaliculus of the eye, in particular into the superior and/or inferior vertical canaliculus of the eye. Due to the intracanalicular localization of the insert, the insert blocks tear drainage through lacrimal occlusion such as also observed for intracanalicular plugs. The intracanalicular inserts of the present invention may be inserted bilaterally or unilaterally into the inferior and/or superior vertical canaliculi of the eyes. According to certain embodiments of the present invention, the intracanalicular insert is a sustained release biodegradable insert.

The terms “API”, “active (pharmaceutical) ingredient”, “active (pharmaceutical) agent”, “active (pharmaceutical) principle”, “(active) therapeutic agent”, “active”, and “drug” are used interchangeably herein and refer to the substance used in a finished pharmaceutical product (FPP) as well as the substance used in the preparation of such a finished pharmaceutical product, intended to furnish pharmacological activity or to otherwise have direct effect in the diagnosis, cure, mitigation, treatment or prevention of a disease, or to have direct effect in restoring, correcting or modifying physiological functions in a patient.

### Nepafenac Drug Substance

Nepafenac is a prodrug of amfenac, used in eye drops to treat pain and inflammation following cataract surgery. Other potential indications include treatment of associated pain and inflammation associated following various surgical refractive procedures, the prevention and treatment of cystoid macular edema (CME), the treatment of allergic conjunctivitis. treatment of central serous chorioretinopathy (CSCR), maintaining intraoperative pupil dilation, preventing/inhibition of intraoperative miosis, and reduction of discomfort and photophobia.

The IUPAC name of nepafenac (INN/USAN) is 2-(2-amino-3-benzoylphenyl)acetamide and the CAS number is 78281-72-8

The molecular formula is C15H14N2O2 and the chemical structure is

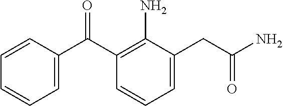 [SMILES: NC(=O)Cc1cccc(C(=O)c2ccccc2)c1N]

Nepafenac has a role as a prodrug, a cyclooxygenase 2 inhibitor, a cyclooxygenase 1 inhibitor, a non-steroidal anti-inflammatory drug (NSAID) and a non-narcotic analgesic. After penetrating the cornea, nepafenac undergoes rapid bioactivation to amfenac (see image below), which is a potent NSAID that uniformly inhibits the COX1 and COX2 activity.

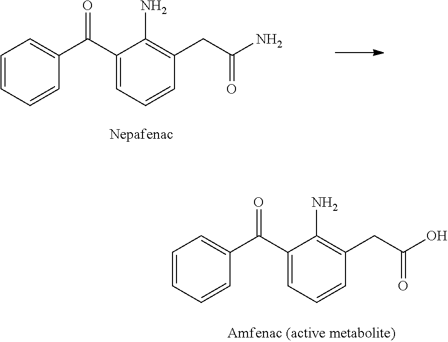 [SMILES: NC(=O)Cc1cccc(C(=O)c2ccccc2)c1N ; Nc1c(CC(=O)O)cccc1C(=O)c1ccccc1]

Nepafenac is a monocarboxylic acid amide that is amfenac in which the carboxylic acid group has been converted into the corresponding carboxamide. Nepafenac is yellow crystalline or powdery substance practically insoluble in water (14 mcg/mL). Nepafenac is soluble in organic solvents such as ethanol, DMSO, and dimethyl formamide (DMF). The solubility of nepafenac in ethanol is approximately 0.5 mg/mL and approximately 30 mg/ml in DMSO and DMF. An aqueous suspension at 0.1% gives an average pH of 6.75. The coefficient partition n-octanol/water is 128. This substance melts at approximately 185° C. and it does not show polymorphism. Nepafenac is an achiral substance and there are no possible variations in the stereochemical configuration.

Nepafenac solubility testing in PBS, pH 7.2 at 37° C. is approximately 102 mcg/mL.

In certain embodiments, for any nonsteroidal anti-inflammatory used in the present invention, including nepafenac, particle sizes (e.g., as expressed by the d90 value) of about 100 μm or below, or of about 75 μm or below, or of about 50 μm or below may be used. In particular embodiments of the present invention, nepafenac may be used in the form of micronized particles and may have a d90 particle size of equal to or less than about 100 μm, or of equal to or less than about 75 μm, or of equal to or less than about 50 μm, or of equal to or less than about 20 μm, or of equal to or less than about 10 μm, or of equal to or less than about 5 μm. In these and other embodiments, the d98 particle size of the micronized nepafenac may be equal to or less than about 100 μm, or equal to or less than about 75 μm, or equal to or less than about 50 μm, or equal to or less than about 25 μm, or equal to or less than about 20 μm, or equal to or less than about 10 μm, or equal to or less than about 5 μm. In particular embodiments of the present invention, the micronized nepafenac has a d90 particle size of equal to or less than about 5 μm and a d98 particle size of less than about 10 μm. The “d90” value means that at least 90 volume-% of all particles within the measured bulk material (which has a certain particle size distribution) has a particle size below the indicated value. For example, a d90 particle size of less than about 50 μm means that at least 90 volume-% of the particles in the measured bulk material have a particle size below about 50 μm. Corresponding definitions apply to other “d” values, such as the “d98” value. The particle size distribution can be measured by methods known in the art, including sieving, laser diffraction or dynamic light scattering. In embodiments in which another nonsteroidal anti-inflammatory than nepafenac is used in the present invention similar particle sizes may apply as disclosed for nepafenac. In certain embodiments, the nepafenac has a d50 of from about 1 μ to about 15 μm, from about 2 μm to about 12 μm, from about 4 μm to about 10 μm or from about 5 μm to about 8 μm In other embodiments, the nepafenac has a d90 of from about 5 μm to about 50 μm, from about 10 μm to about 40 μm, from about 15 μm to about 30 μm or from about 18 μm to about 28 μm. Particles sized can be measure by, e.g., laser diffraction.

For the purposes of certain embodiments of the present invention, in order to increase content uniformity in the final product (and thus to avoid a too high or too low drug content due to discrete particles) and/or reduce potential agglomeration of discrete API particles during manufacturing of the insert (such as during casting the hydrogel as disclosed herein) it may be useful to ensure—in addition to fulfilling a certain particle size specification (e.g. d90 and/or d98 value as disclosed herein)—that there or no or essentially no discrete particles present in the API starting material that are larger than a certain size, such as larger than about 120 μm, or larger than about 100 μm, or larger than about 90 μm. This can be achieved for example by sieving. In particular embodiments, the nepafenac used for manufacturing the inserts according to the present invention has a d90 particle size of equal to or less than about 10 or 5 μm, and/or a d98 particle size of less than about 10 μm, with all or essentially all discrete particles having a size of less than about 90 μm.

For the purposes of the present invention, active agents (including nepafenac) in all their possible forms, including any active agent polymorphs or any pharmaceutically acceptable salts, anhydrates, hydrates, other solvates or derivatives of active agents, can be used. Whenever in this description or in the claims an active agent is referred to by name, e.g., “nepafenac”, even if not explicitly stated, it also refers to any such pharmaceutically acceptable polymorphs, salts, anhydrates, solvates (including hydrates) or derivatives of the active agent. Particularly, the term “nepafenac” refers to nepafenac and pharmaceutically acceptable salts thereof, which may all be used for the purposes of the present invention. The term “polymorph” as used herein refers to any crystalline form of an active agent such as nepafenac. Frequently, active agents that are solid at room temperature exist in a variety of different crystalline forms, i.e., polymorphs, with one polymorph being the thermodynamically most stable at a given temperature and pressure.

As used herein, the term “therapeutically effective” refers to the amount of drug or active agent (i.e., nonsteroidal anti-inflammatory) required to produce a desired therapeutic response or result after administration. For example, in the context of the present invention, one desired therapeutic result would be the reduction of symptoms associated with pain and inflammation.

The term “patient” herein includes both human and animal patients. The inserts according to the present invention are generally suitable for human or veterinary medicinal applications. The patients enrolled and treated in a clinical study may also be referred to as “subjects”. Generally, a “subject” is a (human or animal) individual to which an insert according to the present invention is administered, such as during a clinical study. A “patient” is a subject in need of treatment due to a particular physiological or pathological condition.

The term “average” as used herein refers to a central or typical value in a set of data (points), which is calculated by dividing the sum of the data (points) in the set by their number (i.e., the mean value of a set of data).

As used herein, the term “about” in connection with a measured quantity refers to the normal variations in that measured quantity, as expected by one of ordinary skill in the art in making the measurement and exercising a level of care commensurate with the objective of measurement and the precision of the measuring equipment.

As used herein, the term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that.

As used herein, the singular forms “a,” “an”, and “the” include plural references unless the context clearly indicates otherwise.

The term “and/or” as used in a phrase such as “A and/or B” herein is intended to include both “A and B” and “A or B”.

Open terms such as “include,” “including,” “contain,” “containing” and the like as used herein mean “comprising” and are intended to refer to open-ended lists or enumerations of elements, method steps, or the like and are thus not intended to be limited to the recited elements, method steps or the like but are intended to also include additional, unrecited elements, method steps or the like.

The term “up to” when used herein together with a certain value or number is meant to include the respective value or number. For example, the term “up to 25 days” means “up to and including 25 days”.

The abbreviation “PBS” when used herein means phosphate-buffered saline.

The abbreviation “PEG” when used herein means polyethylene glycol.

All references disclosed herein are hereby incorporated by reference in their entireties for all purposes (with the instant specification prevailing in case of conflict).

### DETAILED DESCRIPTION

In certain embodiments, the invention is directed to a nepafenac intracanalicular insert that is a resorbable hydrogel intracanalicular insert designed for sustained release of nepafenac. In certain embodiments, the insert is intended for punctum placement, to provide sustained topical delivery of nepafenac to the ocular surface for the treatment of pain and inflammation associated with cataract surgery. In certain embodiments, the present invention may be utilized for indications such as treatment of central serous chorioretinopathy (CSCR), maintaining intraoperative pupil dilation, preventing/inhibition of intraoperative miosis, reduction of discomfort and photophobia, treatment of cystoid macular edema (CME) and allergic conjunctivitis.

In certain embodiments, the insert comprises two main components: nepafenac and polyethylene glycol (PEG) based hydrogel covalently conjugated with fluorescein. The fluorescent PEG hydrogel illuminates when excited with a blue light source and aided with a yellow filter to allow for visualization to confirm insert presence. In certain embodiments, nepafenac is embedded in the fluorescent hydrogel matrix of the insert, e.g., in micronized form. In certain embodiments, the dried insert (e.g., in cylindrical form) following administration into the canaliculus will hydrate upon contact with tear fluid causing it to decrease in length and expand in diameter affording retention in the canaliculus. In certain embodiments, the micronized nepafenac gradually dissolves in tear fluid to provide a sustained release of nepafenac over the duration of therapy. In certain embodiments, through hydrolysis, the insert softens, liquefies over time, and is cleared through the nasolacrimal duct without the need for removal.

In certain embodiments, the drug release rate is controlled by drug solubility in the hydrogel matrix predominantly at the interface of tear fluid lavage over the exposed cross-sectional area facing the punctum opening. The insert may contain approximately 0.1 mg to 0.8 mg of nepafenac. Lower doses of nepafenac are intended for shorter durations of drug release and higher doses are intended for longer durations of release.

In certain embodiments, the hydrogel is completely synthetic, with no animal or human derived components. In certain embodiments, the main component of the hydrogel is PEG, which has a long history of safe use in medical devices, pharmaceuticals, and cosmetic products. Nepafenac is also the active ingredient in NEVANAC® (nepafenac ophthalmic suspension, 0.1%) approved in 2005 [NDA #021862].

### The Insert

The present invention generally relates to a sustained release biodegradable ocular insert comprising a hydrogel and a nonsteroidal anti-inflammatory, wherein nonsteroidal anti-inflammatory particles are dispersed within the hydrogel. In certain embodiments, the insert is for administration into the canaliculus of the eye, i.e., is an intracanalicular insert.

The present invention in one aspect relates to a sustained release biodegradable ocular (such as intracanalicular) insert comprising a hydrogel and a nonsteroidal anti-inflammatory, wherein nonsteroidal anti-inflammatory particles are dispersed within the hydrogel, and wherein the insert in its dry state has a length of less than about 3.5 mm, less than about 3.2 mm, less than about 3.0 mm or less than about 2.75 mm.

The present invention in another aspect generally relates to a sustained release biodegradable ocular (such as intracanalicular) insert comprising a hydrogel and equal to or less than about 800 μg nepafenac or an equivalent dose of another nonsteroidal anti-inflammatory.

The present invention in another aspect generally relates to a sustained release biodegradable ocular (such as intracanalicular) insert comprising a hydrogel and a nonsteroidal anti-inflammatory, wherein the insert in a dry state (such as prior to being administered) has a length of equal to or less than about 3.5 mm, less than about 3.2 mm, less than about 3.0 mm or less than about 2.75 mm.

The present invention in another aspect generally relates to a sustained release biodegradable ocular (such as intracanalicular) insert comprising a hydrogel and a nonsteroidal anti-inflammatory, wherein the insert provides for a release of a therapeutically effective amount of the nonsteroidal anti-inflammatory for a period of up to about 7 days, up to about 14 days, up to about 30 days or up to about 42 days after administration.

In all these aspects, a particular nonsteroidal anti-inflammatory for use in the present invention is nepafenac.

In all these aspects, the ocular insert in certain embodiments of the invention may be an intracanalicular insert, i.e., the insert is for insertion/administration into the canaliculus of one or both eye(s).

Each of the features recited above in the three aspects of the invention may be present in a sustained release biodegradable insert of the invention separately, or any two of these features may be present in combination, or all three of these features may be present in combination.

Thus, in one specific embodiment the present invention relates to a sustained release biodegradable intracanalicular insert comprising a hydrogel and nepafenac as the nonsteroidal anti-inflammatory, wherein the insert comprises equal to or less than about 800 μg nepafenac, has a length of equal to or less than about 3.2 mm and provides for a release of a therapeutically effective amount of nepafenac for a period of up to about 14 days after administration.

In certain embodiments, the sustained release biodegradable ocular insert has an average nepafenac release rate at day 1 of from about 2% to about 25%, from about 5% to about 20%, from about 10% to about 18%, about 3% to about 18% or about 3% to about 10% in 1× phosphate buffered saline (PBS) (pH 7.4) at 37 C.

In certain embodiments, the sustained release biodegradable ocular insert has an average nepafenac release rate at day 2 of from about 5% to about 50%, from about 8% to about 40%, from about 15% to about 35%, from about 15% to about 25% or from about 25% to about 35% in 1×PBS (pH 7.4) at 37 C.

In certain embodiments, the sustained release biodegradable ocular insert has an average nepafenac release rate at day 9 of from about 65% to about 100%, from about 75% to about 98% or from about 80% to about 95% in 1×PBS (pH 7.4) at 37 C.

Specific embodiments and features of the insert of the present invention are disclosed below.

### The Active Principle

The present invention in certain embodiments generally relates to a sustained release biodegradable ocular (such as an intracanalicular) insert comprising a hydrogel and a nonsteroidal anti-inflammatory. One particular nonsteroidal anti-inflammatory for use in all aspects of the present invention is nepafenac. Details on nepafenac, its chemical structure and its properties such as solubility are disclosed herein in the definitions section.

In particular embodiments of the present invention the nonsteroidal anti-inflammatory contained in a sustained release biodegradable ocular (such as intracanalicular) insert is nepafenac, and is present in the insert in a range of doses, from about 100 μg to about 1200 μg, or about 10 μg to about 800 μg, or from about 50 μg to about 600 μg. Any nepafenac amount within these dose ranges may be used, such as about 50 μg, about 100 μg, about 150 μg, about 400 μg, about 450 μg, about 500 μg, about 600 μg etc., all values also including a variance of +25% and −20%, or a variance of ±10%. In certain particular embodiments, the doses of nepafenac contained in an insert of the invention are:

The disclosed amounts of nonsteroidal anti-inflammatory, such as nepafenac, including the mentioned variances, refer to both the final content of the active principle in the insert, as well as to the amount of active principle used as a starting component when manufacturing the insert.

In certain embodiments, the nonsteroidal anti-inflammatory, such as nepafenac, may be contained in the insert of the invention such that particles of the nonsteroidal anti-inflammatory are dispersed or distributed in a hydrogel comprised of a polymer network. In certain embodiments, the particles are homogeneously dispersed in the hydrogel. The hydrogel may prevent the drug particles from agglomerating and may provide a matrix for the particles which releases the drug in a sustained manner upon contact with the tear fluid.

In certain embodiments of the invention, the nonsteroidal anti-inflammatory particles, such as the nepafenac particles, may be microencapsulated. The term “microcapsule” is sometimes defined as a roughly spherical particle with a size varying between e.g., about 50 nm to about 2 mm. Microcapsules have at least one discrete domain (or core) of active agent encapsulated in a surrounding or partially surrounding material, sometimes also referred to as a shell. A suitable agent for microencapsulating the nonsteroidal anti-inflammatory, such as the nepafenac, for the purposes of the present invention, is poly(lactic-co-glycolic acid).

In one embodiment, the nonsteroidal anti-inflammatory particles, such as the nepafenac particles, may have a small particle size and may be micronized particles. In another embodiment, the nonsteroidal anti-inflammatory particles, such as the nepafenac particles, may not be micronized. Micronization refers to the process of reducing the average diameter of particles of a solid material. Particles with reduced diameters may have inter alia higher dissolution rates, which increases the bioavailability of active pharmaceutical ingredients. In the composite materials field, particle size is known to affect the mechanical properties when combined with a matrix, with smaller particles providing superior reinforcement for a given mass fraction. Thus, a hydrogel matrix within which micronized nonsteroidal anti-inflammatory particles are dispersed may have improved mechanical properties (e.g., brittleness, strain to failure, etc.) compared to a similar mass fraction of larger nonsteroidal anti-inflammatory particles. Such properties are important in manufacturing, during administration, and during degradation of the insert. Micronization may also promote a more homogeneous distribution of the active ingredient in the chosen dosage form or matrix. In certain embodiments, for any nonsteroidal anti-inflammatory used in the present invention, including nepafenac, particle sizes (e.g., as expressed by the d90 value as defined herein and that are measured as also disclosed herein) of about 100 μm or below, or of about 75 μm or below, or of about 50 μm or below may be used. In particular embodiments, nepafenac may be used in the form of micronized particles and may have a d90 particle size of equal to or less than about 100 μm, or of equal to or less than about 75 μm, or of equal to or less than about 50 μm, or of equal to or less than about 20 μm, or of equal to or less than about 10 μm, or of equal to or less than about 5 μm. In these and other embodiments, the d98 particle size of the micronized nepafenac may be equal to or less than about 100 μm, or equal to or less than about 75 μm, or equal to or less than about 50 μm, or equal to or less than about 25 μm, or equal to or less than about 20 μm, or equal to or less than about 10 μm, or equal to or less than about 5 μm. In particular embodiments of the present invention, the micronized nepafenac used in (or used for preparing) an insert of the present invention has a d90 particle size of equal to or less than about 5 μm and a d98 particle size of less than about 10 μm. In embodiments in which another nonsteroidal anti-inflammatory than nepafenac is used in the present invention similar particle sizes may apply as disclosed for nepafenac.

In certain embodiments, to reduce the presence of discrete particles in the nonsteroidal anti-inflammatory, in particular the nepafenac, starting material that are larger than a certain size, such as larger than about 120 μm, or larger than about 100 μm, or larger than about 90 μm, the bulk nonsteroidal anti-inflammatory material meeting the (d90 and/or d98) particle size specification(s) as disclosed herein may be sieved prior to preparing the wet composition of the insert. In particular embodiments, the nepafenac used for manufacturing the inserts according to the present invention has a d90 particle size of equal to or less than about 5 μm, and a d98 particle size of less than about 10 μm, with all or essentially all discrete particles having a size of less than about 90 μm.

### The Polymer Network

In certain embodiments, the hydrogel may be formed from precursors having functional groups that form crosslinks to create a polymer network. These crosslinks between polymer strands or arms may be chemical (i.e., may be covalent bonds) and/or physical (such as ionic bonds, hydrophobic association, hydrogen bridges etc.) in nature.

The polymer network may be prepared from precursors, either from one type of precursor or from two or more types of precursors that are allowed to react. Precursors are chosen in consideration of the properties that are desired for the resultant hydrogel. There are various suitable precursors for use in making the hydrogels. Generally, any pharmaceutically acceptable and crosslinkable polymers forming a hydrogel may be used for the purposes of the present invention. The hydrogel and thus the components incorporated into it, including the polymers used for making the polymer network, should be physiologically safe such that they do not elicit e.g., an immune response or substantial immune response or other adverse effects. Hydrogels may be formed from natural, synthetic, or biosynthetic polymers.

Natural polymers may include glycosaminoglycans, polysaccharides (e.g., dextran), polyaminoacids and proteins or mixtures or combinations thereof, while this list is not intended to be limiting.

Synthetic polymers may generally be any polymers that are synthetically produced from a variety of feedstocks by different types of polymerization, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts. Synthetic polymers may in certain embodiments be used to lower the potential of allergies in dosage forms that do not contain any ingredients from human or animal origin.

Generally, for the purposes of the present invention one or more synthetic polymers of the group comprising one or more units of polyalkylene glycol, particularly including but not limited to polyethylene glycol (PEG), polyalkylene oxide such as polyethylene oxide, polypropylene oxide, polyvinyl alcohol, poly (vinylpyrrolidinone), polylactic acid, polylactic-co-glycolic acid, random or block copolymers or combinations/mixtures of any of these can be used, while this list is not intended to be limiting.

To form covalently crosslinked polymer networks, the precursors may be covalently crosslinked with each other. In certain embodiments, precursors with at least two reactive centers (for example, in free radical polymerization) can serve as crosslinkers since each reactive group can participate in the formation of a different growing polymer chain.

The precursors may have biologically inert and hydrophilic portions, e.g., a core. In the case of a branched polymer, a core refers to a contiguous portion of a molecule joined to arms that extend from the core, where the arms carry a functional group, which is often at the terminus of the arm or branch. Multi-armed PEG precursors are examples of such precursors and are used in particular embodiments of the present invention as further disclosed herein.

A hydrogel for use in the present invention can be made e.g., from one multi-armed precursor with a first (set of) functional group(s) and another (e.g. multi-armed) precursor having a second (set of) functional group(s). By way of example, a multi-armed precursor may have hydrophilic arms, e.g., polyethylene glycol units, terminated with primary amines (nucleophile), or may have activated ester end groups (electrophile). The polymer network according to the present invention may contain identical or different polymer units crosslinked with each other. The precursors may be high-molecular weight components (such as polymers having functional groups as further disclosed herein) or low-molecular weight components (such as low-molecular amines, thiols, esters etc. as also further disclosed herein).

Certain functional groups can be made more reactive by using an activating group. Such activating groups include (but are not limited to) carbonyldiimidazole, sulfonyl chloride, aryl halides, sulfosuccinimidyl esters, N-hydroxysuccinimidyl (abbreviated as “NHS”) ester, succinimidyl ester, benzotriazolyl ester, thioester, epoxide, aldehyde, maleimides, imidoesters, acrylates and the like. The NHS esters are useful groups for crosslinking with nucleophilic polymers, e.g., primary amine-terminated or thiol-terminated polyethylene glycols or other nucleophilic group-containing agents, such as nucleophilic group-containing crosslinking agents. An NHS-amine crosslinking reaction may be carried out in aqueous solution and in the presence of buffers, e.g., phosphate buffer (pH 5.0-7.5), triethanolamine buffer (pH 7.5-9.0), borate buffer (pH 9.0-12), or sodium bicarbonate buffer (pH 9.0-10.0).

In certain embodiments, each precursor may comprise only nucleophilic or only electrophilic functional groups, so long as both nucleophilic and electrophilic precursors are used in the crosslinking reaction. Thus, for example, if a crosslinker has only nucleophilic functional groups such as amines, the precursor polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly (allyl amine), or amine-terminated di- or multifunctional poly (ethylene glycol) can be also used to prepare the polymer network of the present invention.

In one embodiment of the present invention a precursor for the polymer network forming the hydrogel in which the nonsteroidal anti-inflammatory is dispersed to form the insert according to the present invention has about 2 to about 16 nucleophilic functional groups each (termed functionality), and in another embodiment a precursor has about 2 to about 16 electrophilic functional groups each (termed functionality). Reactive precursors having a number of reactive (nucleophilic or electrophilic) groups as a multiple of 4, thus for example 4, 8 and 16 reactive groups, are particularly suitable for the present invention. However, any number of functional groups, such as including any of 2, 3, 4, 5, 6, 7, 8, 9, 10, 11, 12, 13, 14, 15, or 16 groups, is possible for precursors to be used in accordance with the present invention, while ensuring that the functionality is sufficient to form an adequately crosslinked network.

### PEG Hydrogels

In certain embodiments of the present invention, the polymer network forming the hydrogel contains polyethylene glycol (“PEG”) units. PEGs are known in the art to form hydrogels when crosslinked, and these PEG hydrogels are suitable for pharmaceutical applications e.g., as matrix for drugs intended to be administered to any part of the human or animal body.

The polymer network of the hydrogel inserts of the present invention may comprise one or more multi-arm PEG units having from 2 to 10 arms, or from 4 to 8 arms, or 4, 5, 6, 7 or 8 arms. In certain embodiments, the PEG units used in the hydrogel of the present invention have 4 arms. In certain embodiments, the PEG units used in the hydrogel of the present invention have 8 arms. In certain embodiments, PEG units having 4 arms and PEG units having 8 arms are used in the hydrogel of the present invention. In certain particular embodiments, one or more 4-armed PEGs is/are utilized. Any combination of multi-armed PEGs may be used. In specific embodiments, only 4-arm PEG units are used (which may be the same or different).

The number of arms of the PEG(s) used contributes to controlling the flexibility or softness of the resulting hydrogel. For example, hydrogels formed by crosslinking 4-arm PEGs are generally softer and more flexible than those formed from 8-arm PEGs of the same molecular weight. In particular, if stretching the hydrogel prior to (or also after) drying as disclosed herein below in the section relating to the manufacture of the insert is desired, a more flexible hydrogel may be used, such as a 4-arm PEG, optionally in combination with another multi-arm PEG, such as an 8-arm PEG as disclosed above, or another (different) 4-arm PEG.

In certain embodiments of the present invention, polyethylene glycol units used as precursors have an average molecular weight (Mn) in the range from about 2,000 to about 100,000 Daltons, or in a range from about 10,000 to about 60,000 Daltons, or in a range from about 15,000 to about 50,000 Daltons. In certain particular embodiments the polyethylene glycol units have an average molecular weight in a range from about 10,000 to about 40,000 Daltons, or in a range from about 15,000 to about 30,000 Daltons, or in a range from about 15,000 to about 25,000 Daltons. In specific embodiments, the polyethylene glycol units used for making the hydrogels according to the present invention have an average molecular weight (Mn) of about 20,000 Daltons. Polyethylene glycol precursors of different molecular weight may be combined with each other. When referring herein to a PEG material having a particular average molecular weight (as defined herein), such as about 20,000 Daltons, a variance of ±10% is intended to be included, i.e., referring to a material having an average molecular weight of about 20,000 Daltons also refers to such a material having an average molecular weight of about 18,000 to about 22,000 Daltons. As used herein, the abbreviation “k” in the context of the molecular weight refers to 1,000 Daltons, i.e., “20 k” means 20,000 Daltons.

Further, when referring to a PEG precursor having a certain average molecular weight, such as a 15 kPEG- or a 20 kPEG-precursor, the indicated average molecular weight (i.e., a Mn of 15,000 or 20,000, respectively) refers to the PEG part of the precursor, before end groups are added (“20 k” here means 20,000 Daltons, and “15 k” means 15,000 Daltons-the same abbreviation is used herein for other average molecular weights of PEG precursors). In certain embodiments, the Mn of the PEG part of the precursor is determined by MALDI. The degree of substitution with end groups as disclosed herein may be determined by means of ¹H-NMR after end group functionalization.

In a 4-arm (“4a”) PEG, in certain embodiments each of the arms may have an average arm length (or molecular weight) of the total molecular weight of the PEG divided by 4. A 4a20kPEG precursor, which is a particularly suitably precursor for use in the present invention thus has 4 arms with an average molecular weight of about 5,000 Daltons each and a total molecular weight of 20,000 Daltons. An 8a20k PEG precursor, which could also be used in combination with or alternatively to the 4a20kPEG precursor in the present invention, thus has 8 arms (“8a”) each having an average molecular weight of 2,500 Daltons and a total molecular weight of 20,000 Daltons. Longer arms may provide increased flexibility as compared to shorter arms. PEGs with longer arms may swell more as compared to PEGs with shorter arms. A PEG with a lower number of arms also may swell more and may be more flexible than a PEG with a higher number of arms. In certain particular embodiments, only one or more 4-arm PEG precursor(s) is/are utilized in the present invention. In certain other embodiments, a combination of one or more 4-arm PEG precursor(s) and one or more 8-arm PEG precursor(s) is utilized in the present invention. In addition, longer PEG arms have higher melting temperatures when dry, which may provide more dimensional stability during storage.

In certain embodiments, electrophilic end groups for use with PEG precursors for preparing the hydrogels of the present invention are N-hydroxysuccinimidyl (NHS) esters, including but not limited to NHS dicarboxylic acid esters such as the succinimidylmalonate group, succinimidylmaleate group, succinimidylfumarate group, “SAZ” referring to a succinimidylazelate end group, “SAP” referring to a succinimidyladipate end group, “SG” referring to a succinimidylglutarate end group, and “SS” referring to a succinimidylsuccinate end group. Examples of other activated esters in addition to the NHS esters that are useful in the present invention are (without being limited to these) thioesters, benzotriazolyl esters, and esters of acrylic acids.

In certain embodiments, nucleophilic end groups for use with electrophilic group-containing PEG precursors for preparing the hydrogels of the present invention are amine (denoted as “NH₂”) end groups. Thiol (—SH) end groups or other nucleophilic end groups are also possible.

In certain embodiments of the present invention, 4-arm PEGs with an average molecular weight of about 20,000 Daltons and electrophilic end groups as disclosed above (such as the SAZ, SAP, SG and SS end groups, particularly the SG end group) are crosslinked for forming the polymer network and thus the hydrogel according to the present invention. Suitable PEG precursors are available from a number of suppliers, such as Jenkem Technology and others.

Reactions of e.g., nucleophilic group-containing crosslinkers and electrophilic group-containing PEG units, such as reaction of amine group-containing crosslinkers with activated ester-group containing PEG units, result in a plurality of PEG units being crosslinked by a hydrolyzable linker having the formula:

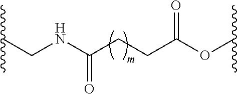

, wherein m is an integer from 0 to 10, and specifically is 1, 2, 3, 4, 5, 6, 7, 8, 9, or 10. For a SAZ-end group, m would be 6, for a SAP-end group, m would be 3, for a SG-end group, m would be 2 and for an SS-end group, m would be 1. In particular embodiments, m is 2. All crosslinks within the polymer network may be the same, or may be different.

In certain embodiments, the polymer precursors used for forming the hydrogel according to the present invention may be selected from 4a20kPEG-SAZ, 4a20kPEG-SAP, 4a20kPEG-SG, 4a20kPEG-SS, 8a20kPEG-SAZ, 8a20kPEG-SAP, 8a20kPEG-SG, 8a20kPEG-SS, or mixtures thereof, with one or more PEG- or lysine based-amine groups selected from 4a20kPEG-NH₂, 8a20 kPEG-NH₂, and trilysine, or a trilysine salt or derivative, such as trilysine acetate.

In certain embodiments, the SG end group is utilized in the present invention. This end group may provide for a shorter time until the hydrogel is biodegraded in an aqueous environment such as in the tear fluid, when compared to the use of other end groups, such as the SAZ end group, which provides for a higher number of carbon atoms in the linker and may thus be more hydrophobic and therefore less prone to ester hydrolysis than the SG end group.

In particular embodiments, a 4-arm 20,000 Dalton PEG precursor having a SG end group (as defined above), is crosslinked with a crosslinking agent having one or more reactive amine end groups. This PEG precursor is abbreviated herein as 4a20kPEG-SG. A schematic chemical structure of 4a20kPEG-SG is reproduced below:

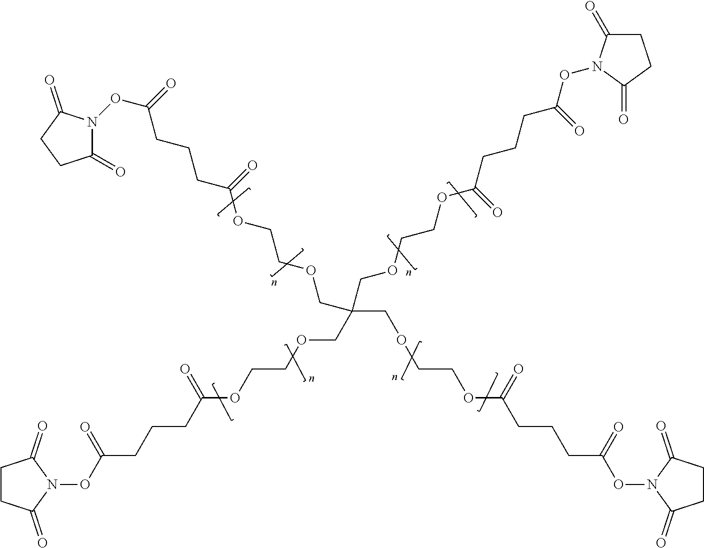

In this formula, n is determined by the molecular weight of the respective PEG-arm.

In certain particular embodiments, the crosslinking agent (herein also referred to as “crosslinker”) used is a low-molecular weight component containing nucleophilic end groups, such as amine or thiol end groups. In certain embodiments, the nucleophilic group-containing crosslinking agent is a small molecule amine with a molecular weight below 1,000 Da. In certain embodiments, the nucleophilic-group containing crosslinking agent comprises two, three or more primary aliphatic amine groups. Suitable crosslinking agents for use in the present invention are (without being limited to) spermine, spermidine, lysine, dilysine, trilysine, tetralysine, polylysine, ethylenediamine, polyethylenimine, 1,3-diaminopropane, 1,3-diaminopropane, diethylenetriamine, trimethylhexamethylenediamine, 1,1,1-tris(aminoethyl)ethane, their pharmaceutically acceptable salts, hydrates or other solvates and their derivatives such as conjugates (as long as sufficient nucleophilic groups for crosslinking remain present), and any mixtures thereof. A particular crosslinking agent for use in the present invention is a lysine-based crosslinking agent, such as trilysine or a trilysine salt or derivative. A particular nucleophilic crosslinking agent for use in the present invention is trilysine acetate. Other low-molecular weight multi-arm amines may be used as well. The chemical structure of trilysine is reproduced below:

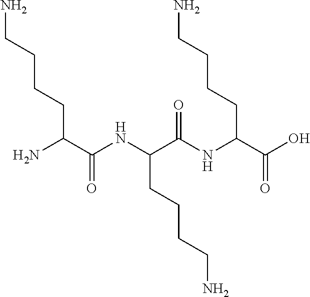 [SMILES: NCCCCC(N)C(=O)NC(CCCCN)C(=O)NC(CCCCN)C(=O)O]

In very particular embodiments of the present invention, a 4a20kPEG-SG precursor is reacted with trilysine acetate, to form the polymer network.

In certain embodiments, the nucleophilic group-containing crosslinking agent is bound to or conjugated with a visualization agent. Fluorophores such as fluorescein, rhodamine, coumarin, and cyanine can be used as visualization agents as disclosed herein. In specific embodiments of the present invention, fluorescein is used as the visualization agent. The visualization agent may be conjugated with the crosslinking agent e.g., through some of the nucleophilic groups of the crosslinking agent. Since a sufficient amount of the nucleophilic groups are necessary for crosslinking, “conjugated” or “conjugation” in general includes partial conjugation, meaning that only a part of the nucleophilic groups may be used for conjugation with the visualization agent, such as about 1% to about 20%, or about 5% to about 10%, or about 8% of the nucleophilic groups of the crosslinking agent may be conjugated with a visualization agent. In specific embodiments, the crosslinking agent is trilysine acetate and is conjugated with fluorescein.

In other embodiments, the visualization agent may also be conjugated with the polymer precursor, e.g. through certain reactive (such as electrophilic) groups of the polymer precursors. In certain embodiments, the crosslinking agent itself or the polymer precursor itself may contain an e.g. fluorophoric or other visualization-enabling group.

In the present invention, conjugation of the visualization agent to either the polymer precursor(s) or to the crosslinking agent as disclosed below is intended to keep the visualization agent in the hydrogel while the active agent is released into the tear fluid, thus allowing confirmation of insert presence within the canaliculus by a convenient, non-invasive method.

In certain embodiments, the molar ratio of the nucleophilic and the electrophilic end groups reacting with each other is about 1:1, i.e., one amine group is provided per one electrophilic, such as SG, group. In the case of 4a20kPEG-SG and trilysine (acetate) this results in a molar ratio of the two components of about 1:1 as the trilysine has four primary amine groups that may react with the electrophilic SG ester group. However, an excess of either the electrophilic (e.g. NHS, such as the SG) end group precursor or of the nucleophilic (e.g. the amine) end group precursor may be used. In particular, an excess of the nucleophilic, such as the amine end group containing precursor or crosslinking agent may be used. In certain embodiments, the molar ratio of the electrophilic group containing precursor to the nucleophilic group-containing crosslinking agent, such as the molar ratio of 4a20kPEG-SG to trilysine acetate, is from about 1:2 to about 2:1.

Finally, in alternative embodiments the amine linking agent can also be another PEG precursor with the same or a different number of arms and the same or a different arm length (average molecular weight) as the 4a20kPEG-SG, but having terminal amine groups, i.e., 4a20kPEG-NH₂.

### Additional Ingredients

The insert of the present invention may contain, in addition to the polymer units forming the polymer network as disclosed above and the active principle, other additional ingredients. Such additional ingredients are for example salts originating from buffers used during the preparation of the hydrogel, such as phosphates, borates, bicarbonates, or other buffer agents such as triethanolamine. In certain embodiments of the present invention sodium phosphate buffers (specifically, mono- and dibasic sodium phosphate) are used.

In a specific embodiment, the insert of the present invention is free of anti-microbial preservatives or at least does not contain a substantial amount of anti-microbial preservatives (including, but not limited to benzalkonium chloride (BAK), chlorobutanol, sodium perborate, and stabilized oxychloro complex (SOC)).

In a further specific embodiment, the insert of the present invention does not contain any ingredients of animals or human origin but only contains synthetic ingredients.

In certain embodiments, the inserts of the present invention contain a visualization agent. Visualization agents to be used according to the present invention are all agents that can be conjugated with the components of the hydrogel or can be entrapped within the hydrogel, and that are visible, or may be made visible when exposed e.g. to light of a certain wavelength, or that are contrast agents. Suitable visualization agents for use in the present invention are (but are not limited to) e.g. fluoresceins, rhodamines, coumarins, cyanines, europium chelate complexes, boron dipyromethenes, benzofrazans, dansyls, bimanes, acridines, triazapentalenes, pyrenes and derivatives thereof. Such visualization agents are commercially available e.g. from TCI. In certain embodiments the visualization agent is a fluorophore, such as fluorescein or comprises a fluorescein moiety. Visualization of the fluorescein-containing insert is possible by illumination with blue light and a yellow filter. The fluorescein in the intracanalicular insert illuminates when excited with blue light enabling confirmation of insert presence. In specific embodiments, the visualization agent is conjugated with one of the components forming the hydrogel. For example, the visualization agent, such as fluorescein, is conjugated with the crosslinking agent, such as the trilysine or trilysine salt or derivate (e.g. the trilysine acetate), or with the PEG-component. For example, NHS-fluorescein may be conjugated with trilysine acetate prior to the crosslinking reaction with the PEG precursor(s). Conjugation of the visualization agent prevents the visualization agent from being eluted or released out of the insert. Since a sufficient amount of the nucleophilic groups (at least more than one molar equivalent) are necessary for crosslinking, partial conjugation of the visualization agent with e.g. the crosslinking agent as disclosed above may be performed.

The insert of the present invention may in certain embodiments contain a surfactant. The surfactant may be a non-ionic surfactant. The non-ionic surfactant may comprise a poly (ethylene glycol) chain. Exemplary non-ionic surfactants are poly (ethylene glycol) sorbitan monolaurate commercially available as Tween® (and in particular Tween®20, a PEG-20-sorbitan monolaurate, or Tween®80, a PEG-80-sorbitan monolaurate), poly(ethylene glycol) ester of castor oil commercially available as Cremophor (and in particular Cremophor40, which is PEG-40-castor oil), and an ethoxylated 4-tert-octylphenol/formaldehyde condensation polymer which is commercially available as Tyloxapol and others such as Triton. A surfactant may aid in dispersing the active principle and may prevent particle aggregation, and may also reduce possible adhesion of the hydrogel strand to the tubing during drying.

### Formulation

In certain embodiments, inserts according to the present invention comprise a nonsteroidal anti-inflammatory, such as nepafenac, a polymer network made from one or more polymer precursors as disclosed herein in the form of a hydrogel, and optional additional components such as visualization agents, salts etc. remaining in the insert from the production process (such as phosphate salts used as buffers etc.). In certain preferred embodiments, the nonsteroidal anti-inflammatory is nepafenac. In particular embodiments, the insert is preservative-free.

In some embodiments, the inserts according to the present invention in a dry state contain from about 25% to about 75% by weight nonsteroidal anti-inflammatory, such as nepafenac, and from about 75% to about 25% by weight polymer units, such as those disclosed above. In further embodiments, the inserts according to the present invention in a dry state contain from about 40% to about 70% by weight nonsteroidal anti-inflammatory, such as nepafenac, and from about 20% to about 60% by weight polymer units, such as those disclosed above.

In certain embodiments, the inserts according to the present invention in a dry state contain from about 50% to about 70% by weight nonsteroidal anti-inflammatory, such as nepafenac, and from about 20% to about 60% by weight polymer units, such as polyethylene glycol units as disclosed above.

In certain embodiments, the inserts according to the present invention may contain in a dry state about 0.1% to about 1% by weight visualization agent, such as fluorescein or a molecule comprising a fluorescein moiety. Also in certain embodiments, the inserts according to the present invention may contain in a dry state about 0.5% to about 5% by weight of one or more buffer salt(s) (separately or taken together). In certain embodiments, the insert in a dry state may contain, e.g., from about 0.01% to about 2% by weight or from about 0.05% to about 0.5% by weight of a surfactant.

In certain embodiments, the balance of the insert in its dry state (i.e., the remainder of the formulation when nonsteroidal anti-inflammatory, such as nepafenac, and polymer hydrogel, such as trilysine-crosslinked PEG hydrogel, and optionally visualization agent, such as fluorescein, have already been taken account of) may be salts remaining from the buffer used during manufacture of the inserts as disclosed herein, or may be other ingredients used during manufacturing of the insert (such as surfactants if used). In certain embodiments, such salts are phosphate, borate or (bi) carbonate salts. In one embodiment a buffer salt is sodium phosphate (mono- and/or dibasic).

The amounts of the nonsteroidal anti-inflammatory and the polymer(s) may be varied, and other amounts of the nonsteroidal anti-inflammatory and the polymer hydrogel than those disclosed herein may also be used to prepare inserts according to the invention.

In certain embodiments, solid contents of about 20% to about 50% (w/v) (wherein “solids” means the combined weight of polymer precursor(s), optional visualization agent, salts and the drug in solution) are utilized for forming the hydrogel of the inserts according to the present invention.

In certain embodiments, the water content of the hydrogel in a dry (dehydrated/dried) state may be low, such as not more than about 1% by weight of water (determined e.g. as disclosed herein). The water content may in certain embodiments also be lower than that, possibly no more than about 0.25% by weight or even no more than about 0.1% by weight.

### Dimensions of the Insert and Dimensional Change Upon Hydration

The dried insert may have different geometries, depending on the method of manufacture, such as the inner diameter or shape of a mold or tubing into which the mixture comprising the hydrogel precursors including the nonsteroidal anti-inflammatory is cast prior to complete gelling. The insert according to the present invention is also referred as a “fiber” (which term is used interchangeably herein with the term “rod”), wherein the fiber in general has a length that exceeds its diameter. The insert (or the fiber) may have different geometries, with specific dimensions as disclosed herein.

In one embodiment, the insert is cylindrical or has an essentially cylindrical shape. Whenever in the specification or in the claims it is herein referred to “cylindrical” in the context of the shape of the insert, this always includes “essentially cylindrical”. In this case, the insert has a round or an essentially round cross-section. In other embodiments of the invention, the insert is non-cylindrical. The insert according to the present invention is optionally elongated in its dry state, wherein the length of the insert is greater than the width of the insert, wherein the width is the largest cross-sectional dimension that is substantially perpendicular to the length. In a cylindrical or essentially cylindrical insert, the width is also referred to as the diameter.

Various geometries of the outer insert shape or its cross-section may also be used in the present invention. For example, instead of a round diameter fiber (i.e., in the case of a cylindrical insert), an oval (or elliptical) diameter fiber may be used. Other cross-sectional geometries, such as oval or oblong, rectangular, triangular, star-shaped, cross-shaped etc. may generally be used. As long as the insert expands in diameter upon hydration in the canaliculus to a hydrated diameter as disclosed herein, the exact cross-sectional shape is not decisive, as tissue will form around the insert. In certain embodiments, the ratio of the length of the insert to the diameter of the insert in the hydrated state is at least about 1, or at least about 1.1, or at least about 1.2, or at least about 1.4 or at least about 1.7 which aids in keeping the insert in place in the canaliculus and prevents the insert from twisting and turning within the canaliculus, and also aids in maintaining a close contact with surrounding tissue. In certain embodiments, this ratio may be less than about 2.5, less than about 2.2, less than about 2, or less than about 1.8.

The polymer network, such as the PEG network, of the hydrogel insert according to certain embodiments of the present invention may be semi-crystalline in the dry state at or below room temperature, and amorphous in the wet state. Even in the stretched form, the dry insert may be dimensionally stable at or below room temperature, which may be advantageous for administering the insert into the canaliculus, and also for quality control.

Upon hydration of the insert in the canaliculus by the tear fluid (which can be simulated in vitro e.g. by immersing the insert into PBS, pH 7.4 at 37° C. after 24 hours, which is considered equilibrium) the dimensions of the insert according to the invention may change. Generally, the diameter of the insert may increase, while its length may decrease or in certain embodiments may stay the same or essentially the same. An advantage of this dimensional change is that, while the insert in its dry state is sufficiently thin to be administered and placed into the canaliculus through the punctum (which itself is smaller in diameter than the canaliculus) upon hydration and thereby through expansion of its diameter it fits closely into the canaliculus and thus acts as a canalicular plug. The insert therefore provides for lacrimal occlusion and thereby tear conservation in addition to releasing the active principle in a controlled manner to the tear fluid over a certain period of time as disclosed herein.

In certain embodiments, this dimensional change is enabled at least in part by the “shape memory” effect introduced into the insert by means of stretching the hydrogel strand in the longitudinal direction during its manufacture as also disclosed herein. In certain embodiments, this stretching may be performed in the wet state, i.e., before drying. However, in certain other embodiments, the stretching of the hydrogel strands (once casted and cured) may be performed in the dry state (i.e., after drying the hydrogel strands). It is noted that if no stretching is performed at all the insert may merely swell due to the uptake of water, but the dimensional change of an increase in diameter and a decrease in length disclosed herein may not be achieved, or may not be achieved to a large extent. This could result in a less than optimal fixture of the insert in the canaliculus, and could potentially lead to the insert being cleared (potentially even prior to the release of the complete dose of the active principle) through the nasolacrimal duct or through the punctum. If this is not desired, the hydrogel strand may e.g. be dry or wet stretched in order to provide for expansion of the diameter upon rehydration.

In the hydrogels of the present invention, a degree of molecular orientation may be imparted by stretching the material then allowing it to solidify, locking in the molecular orientation. The molecular orientation provides one mechanism for anisotropic swelling upon contacting the insert with a hydrating medium such as tear fluid. Upon hydration, the insert of certain embodiments of the present invention will swell only in the radial dimension, while the length will either decrease or be maintained or essentially maintained. The term “anisotropic swelling” means swelling preferentially in one direction as opposed to another, as in a cylinder that swells predominantly in diameter, but does not appreciably expand (or does even contract) in the longitudinal dimension.

The degree of dimensional change upon hydration may depend inter alia on the stretch factor. Merely as an example to illustrate the effect of stretching, stretching at e.g. a stretch factor of about 1.3 (e.g. by means of wet stretching) may have a less pronounced effect or may not change the length and/or the diameter during hydration to a large extent. In contrast, stretching at e.g. a stretch factor of about 1.8 (e.g. by means of wet stretching) may result in a shorter length and/or an increased diameter during hydration. Stretching at e.g. a stretch factor of about 3 or 4 (e.g. by means of dry stretching) could result in a much shorter length and a much larger diameter upon hydration. One skilled in the art will appreciate that other factors besides stretching can also affect swelling behavior.

Among other factors influencing the possibility to stretch the hydrogel and to elicit dimensional change of the insert upon hydration is the composition of the polymer network. In the case PEG precursors are used, those with a lower number of arms (such as 4-armed PEG precursors) contribute to providing a higher flexibility in the hydrogel than those with a higher number of arms (such as 8-armed PEG precursors). If a hydrogel contains more of the less flexible components (e.g. a higher amount of PEG precursors containing a larger number of arms, such as the 8-armed PEG units), the hydrogel may be firmer and less easy to stretch without fracturing. On the other hand, a hydrogel containing more flexible components (such as PEG precursors containing a lower number of arms, such as 4-armed PEG units) may be easier to stretch and softer, but also swells more upon hydration. Thus, the behavior and properties of the insert once it has been administered and is rehydrated can be tailored by means of varying structural features as well as by modifying the processing of the insert after it has been initially formed.

The dried insert dimensions inter alia may depend on the amount of nonsteroidal anti-inflammatory incorporated as well as the ratio of nonsteroidal anti-inflammatory to polymer units and can additionally be controlled by the diameter and shape of the mold or tubing in which the hydrogel is allowed to gel. The diameter of the dried insert may be further controlled by (wet or dry) stretching of the hydrogel strands once formed as disclosed herein. The dried hydrogel strands (after stretching) are cut into segments of the desired length to form the insert; the length can thus be chosen as desired.

In the following, embodiments of inserts with specific dimensions are disclosed. The dimensional ranges or values disclosed in the specific embodiments herein relate to the length and the diameter of cylindrical or essentially cylindrical inserts. However, all values and ranges for cylindrical inserts may also be used correspondingly for non-cylindrical inserts. In case several measurements of the length or diameter of one insert are conducted, or several datapoints are collected during the measurement, the average (i.e., mean) value is reported as defined herein. The length and diameter of an insert according to the invention may be measured e.g. by means of microscopy, or by means of an (optionally automated) camera system. Other suitable methods of measuring insert dimensions may also be used.

In one embodiment, the present invention relates to a sustained release biodegradable intracanalicular insert comprising a hydrogel and a nonsteroidal anti-inflammatory, wherein the insert in a dry state has a length of equal to or less than about 3.5 mm, less than about 3.2 mm or less than about 3.00 mm. In a particular embodiment, the nonsteroidal anti-inflammatory is nepafenac.

In certain embodiments of the invention, the insert in a dry state has a length of equal to or less than about 3.5 mm, equal to or less than about 3.2, mm, equal to or less than about 3.0 mm, equal to or less than about 2.8 mm, or less than about 2.6 mm, or has a length of about 2.5 mm. In certain embodiments of the invention, the insert in a dry state has a length of greater than about 1 mm, or greater than about 1.5 mm, or greater than about 2 mm or greater than about 2.5 mm, or greater than about 2.8 mm. In certain particular embodiments, the insert in its dry state has a length of equal to or less than about 3.2 mm and greater than about 2.8 mm.

In alternative embodiments, the insert may have a length of about 0.5 mm to about 3.5 mm (e.g., about 0.5 mm to about 3.2 mm, about 1 mm to about 3.0 mm, about 1.25 mm to about 2.8 mm, about 1.5 mm to about 2.5 mm, about 0.5 mm, about 0.75 mm, about 1 mm, about 1.25 mm, about 1.5 mm, about 1.75 mm, about 2.0 mm, about 2.25 mm, about 2.5 mm, about 2.75 mm, or about 3 mm).

In certain embodiments of the invention, the insert in a dry state has a diameter of less than about 1 mm, or less than about 0.8 mm, or less than about 0.75 mm, or less than about 0.6 mm, or a diameter from about 0.40 mm to about 0.7 mm, or of about 0.5 mm, or of about 0.6 mm.

In certain embodiments, an insert according to the invention is cylindrical or essentially cylindrical and upon hydration (in vivo in the canaliculus, or in vitro after 24 hours in phosphate-buffered saline at a pH of 7.2 at 37° C.) the diameter of the insert is increased and the length of the insert is decreased. In particular, the diameter of the insert may be increased by a factor in the range of about 1.5 to about 4, or of about 2 to about 3.5, or of about 3. In other words, the ratio of the diameter of the insert in the hydrated state to the diameter of the insert in the dry state may be in the range of about 1.5 to about 4, or of about 2 to about 3.5, or of about 3.

In certain embodiments, the length of an insert according to the invention is decreased after hydration to about 0.99 or less or 0.95 times its length in the dry state, 0.92 times its length in the dry state, 0.90 times its length in the dry state or to about 0.75 times its length in the dry state, or to about two-thirds of its length in the dry state. In other words, the ratio of the length of the insert in the hydrated state to the length of the insert in the dry state may be about 0.99 or less, or about 0.95 or less, or about 0.9 or less or about 0.85 or less or about two-thirds or less, and may be at least about 0.25, or at least about 0.4.

Thus, in certain embodiments an insert according to the present invention in its hydrated state has a diameter in the range of about 1 to about 2.5 mm, and a length that is shorter than the length of the insert in its dry state. In certain embodiments, in the hydrated state, e.g. when the insert has been placed into the canaliculus, the ratio of length to diameter of the insert is suitably greater than 1, i.e., the length of the insert is longer than its diameter. This aids in keeping the insert in place in the canaliculus without any twisting or turning. This aids in occluding the canaliculus/the punctum and keeping the tear fluid within the eye, as well as ensuring contact between the surface of the insert and the tear fluid for releasing the nonsteroidal anti-inflammatory such as nepafenac.

In certain embodiments, an insert according to the present invention in the hydrated state has a diameter in the range of about 1.4 mm to about 2.2 mm or about 1.4 mm to about 2.0 mm or about 1.5 mm or about 1.8 mm.

In certain embodiments, the dimensional change may be achieved by wet stretching the hydrogel strand at a stretch factor in the range of about 1.5 to about 3, or of about 2.2 to about 2.8, or of about 2.5 to about 2.6. In other embodiments, such dimensional change may be achieved by dry stretching.

In certain embodiments, the stretching thus creates a shape memory, meaning that the insert upon hydration when administered into the canaliculus and once it comes into contact with the tear fluid, will shrink in length and widen in diameter until it approaches (more or less) its equilibrium dimensions, which are determined inter alia by the original molded dimensions and compositional variables. While the narrow dry dimensions facilitate administration of the insert through the punctum into the canaliculus, the widened diameter and shortened length after administration yield a shorter but wider insert that fits closely into and occludes the canaliculus while releasing active agent primarily at its proximal surface (the surface of the insert that is in contact with the tear fluid and that is directed toward the punctum opening).

In certain embodiments, an insert of the present invention has a total weight in the range of about 50 to about 1500 μg, such as in the range of about 100 to about 1000 μg, or in the range of about 200 to about 800 μg, or in the range of about 400 to about 1100 μg or about 650 to about 1000 μg or about 50 to about 1200 μg.

### Release of the Active and Biodegradation of the Insert

In one embodiment, the present invention relates to a sustained release biodegradable ocular (such as intracanalicular) insert comprising a hydrogel and a nonsteroidal anti-inflammatory, wherein the insert provides for a release of a therapeutically effective amount of the nonsteroidal anti-inflammatory or metabolite for a period of up to about 5 days, up to about 7 days, up to about 14 days up to about 21 days, up to about 30 days or up to about 42 days after administration (i.e., after having been inserted into the canaliculus). In a particular embodiment, the nonsteroidal anti-inflammatory is nepafenac and the metabolite is amfenac.

In certain embodiments, the active agent gradually gets dissolved and diffuses out of the hydrogel into the tear fluid. This happens primarily in a unidirectional manner, starting at the interface of the insert and the tear fluid at the proximal surface of the insert. The “drug front” generally progresses in the opposite direction, i.e., away from the proximal surface until eventually the entire insert is depleted of active agent.

In certain embodiments, after administration, the levels of active agent released from the insert per day remain sustained, constant or essentially constant over a certain period of time (due to the limitation of release based on the active agent's solubility), such as for about 5 days, or about 7 days, or for about 11 days, or for about 14 days in the case of nepafenac. Then the amount of active agent released per day may decrease for another period of time (also referred to as “tapering”), such as for a period of about 7 additional days (or longer in certain embodiments) in the case of nepafenac until all or substantially all of the active agent has been released and the “empty” hydrogel remains in the canaliculus until it is fully degraded and/or until it is cleared (disposed/washed out) through the nasolacrimal duct.

In certain embodiments, when drug is released primarily from the proximal surface of the insert, this region of the hydrogel insert becomes devoid of drug particles and may therefore also be called the “clearance zone”. In certain embodiments, upon hydration the “clearance zone” is thus a region of the insert that has a concentration of active agent that is less than the active agent in another region of the hydrated hydrogel. As the clearance zone increases, it creates a concentration gradient within the insert that may lead to tapering of the release rate of the drug.

In certain embodiments, concurrently with the drug diffusing out of the hydrogel (and also after the entire amount of drug has diffused out of the hydrogel), the hydrogel may be slowly degraded e.g. by means of ester hydrolysis in the aqueous environment of the tear fluid. At advanced stages of degradation, distortion and erosion of the hydrogel begins to occur. As this happens, the hydrogel becomes softer and more liquid (and thus its shape becomes distorted) until the hydrogel finally dissolves and is resorbed completely. However, as the hydrogel becomes softer and thinner and its shape becomes distorted, at a certain point it may no longer remain at its intended site in the canaliculus to which it had been administered, but it may progress deeper into the canaliculus and eventually may be cleared (disposed/washed out) through the nasolacrimal duct.

In one embodiment, the persistence of the hydrogel within an aqueous environment such as in the human eye (including the canaliculus) depends inter alia on the structure of the linker that crosslinks the polymer units, such as the PEG units, in the hydrogel. In certain embodiments, the hydrogel is biodegraded within a period of about 1 week, about 2 weeks, or about 1 month, or about 2 months, or about 3 months, or up to about 4 months, after administration. However, since during the degradation process in the aqueous environment, such as in the tear fluid within the canaliculus, the hydrogel gradually becomes softer and distorted, the insert may be cleared (washed out/disposed) through the nasolacrimal duct before it is completely biodegraded.

In embodiments of the present invention, the hydrogel and thus the insert remains in the canaliculus for a period of up to about 1 week, up to about two weeks, 1 month, or up to about 2 months, or up to about 3 months, or up to about 4 months, after administration.

In certain embodiments of the invention, in the case the nonsteroidal anti-inflammatory is nepafenac, the entire amount of nepafenac may be released prior to the complete degradation of the hydrogel, and the insert may persist in the canaliculus thereafter, for a period of altogether up to about 1 week, up to about 2 weeks, about 1 month after administration, or up to about 2 months after administration, or up to about 3 months, or up to about 4 months, after administration. In certain other embodiments, the hydrogel may be fully biodegraded when the nonsteroidal anti-inflammatory, such as nepafenac, has not yet been completely released from the insert. In other embodiments, the insert may be fully degraded following at least about 90%, or at least about 92%, or at least about 95%, or at least about 97% release of the nonsteroidal anti-inflammatory.

In certain embodiments, in vitro release tests may be used to compare different inserts (e.g. of different production batches, of different composition, and of different dosage strength etc.) with each other, for example for the purpose of quality control or other qualitative assessments. The in vitro-release of a nonsteroidal anti-inflammatory from the inserts of the invention can be determined by various methods, such as under non-sink simulated physiological conditions in PBS (phosphate-buffered saline, pH 7.4) at 37° C., with daily replacement of PBS in a volume comparable to the tear fluid in the human eye.

In certain embodiments, the tear fluid contains nepafenac and amfenac.

In certain embodiments, the ratio of nepafenac to amfenac at 0.1 days in tear fluid is greater than about 2:1; greater than about 3:1; greater than about 4.1 or from about 2:1 to about 6:1 or about 3:1 to about 5:1.

In certain embodiments, the ratio of nepafenac to amfenac at 1 day in tear fluid is greater than about 2:1; greater than about 3:1; greater than about 4.1, greater than about 5:1; greater than about 7:1 or from about 2:1 to about 10:1 or about 6:1 to about 8:1.

In certain embodiments, the ratio of nepafenac to amfenac at 3 days in tear fluid is greater than about 1:1, greater than about 2:1; greater than about 3:1; greater than about 4.1 or from about 2:1 to about 6:1 or about 2:1 to about 5:1.

In certain embodiments, the ratio of nepafenac to amfenac at 7 days in tear fluid is greater than about 1:1; greater than about 1.5:1; greater than about 2:1; greater than about 3:1; greater than about 4.1 or from about 1:1 to about 3:1 or about 1.5:1 to about 2:1.

In certain embodiments, the ratio of nepafenac to amfenac at 10 days in tear fluid is greater than about 1:1, greater than about 2:1; greater than about 3:1; greater than about 4.1 or from about 2:1 to about 6:1 or about 2:1 to about 5:1.

In certain embodiments, the ratio of nepafenac to amfenac at any one of 0.1 days, 1 day, 3 days, 7 days or 10 days in tear fluid is greater than about 1:1, greater than about 2:1; greater than about 3:1; greater than about 4.1; greater than about 5:1; or greater than about 8:1 or from about 1:1 to about 15:1 or about 1:1 to about 10:1.

In certain embodiments, the ratio of amfenac to nepafenac at 5 days, 7 days, 14 days, 21 days, 30 days or 42 days in aqueous humor is greater than about 100:1; greater than about 1000:1; greater than about 10,000:1 or greater than about 100,000:1. In certain embodiments, at 5 days, 7 days, 14 days, 21 days, 30 days or 42 days in aqueous humor, the amfenac is at a concentration compared to the IC50 that is greater than 2 fold, greater than 5 fold, greater than 10 fold, greater than 20 fold, greater than 25 fold, greater than 30 fold, greater than 35 fold, greater than 40 fold, greater than 50 fold, greater than 75 fold, greater than 100 fold, greater than 150 fold, greater than about 200 fold, or from 2 to 500 fold, 5 to 400 fold, from 10 to 300 fold, from 15 to 200 fold, from 20 to 100 fold, from 25 to 75 fold or from 30 to 50 fold.

### Manufacture of the Insert

In certain embodiments, the present invention also relates to a method of manufacturing a sustained release biodegradable intracanalicular insert as disclosed herein, comprising a hydrogel and a nonsteroidal anti-inflammatory, such as nepafenac.

In certain embodiments the method of manufacturing according to the present invention comprises the steps of forming a hydrogel comprising a polymer network (e.g., comprising PEG units) and nonsteroidal anti-inflammatory particles dispersed in the hydrogel, shaping or casting the hydrogel and drying the hydrogel. In one embodiment the nonsteroidal anti-inflammatory, such as nepafenac, may be used in micronized form as disclosed herein for preparing the insert. In another embodiment, the nonsteroidal anti-inflammatory, such as nepafenac, may be used in non-micronized form for preparing the insert.

Suitable precursors for forming the hydrogel of certain embodiments of the invention are as disclosed above in the section relating to the insert itself. In certain specific embodiments, the hydrogel is made of a polymer network comprising crosslinked polyethylene glycol units as disclosed herein. The polyethylene glycol (PEG) units in particular embodiments are multi-arm, such as 4-arm, PEG units having an average molecular weight from about 2,000 to about 100,000 Daltons, or from about 10,000 to about 60,000 Daltons, or from about 15,000 to about 50,000 Daltons, or of about 20,000 Daltons. Suitable PEG precursors having reactive groups such as electrophilic groups as disclosed herein are crosslinked to form the polymer network. Crosslinking may be performed by means of a crosslinking agent that is either a low molecular compound or another polymeric compound, including another PEG precursor, having reactive groups such as nucleophilic groups as also disclosed herein. In certain embodiments, a PEG precursor with electrophilic end groups is reacted with a crosslinking agent (a low-molecular compound, or another PEG precursor) with nucleophilic end groups to form the polymer network.

In specific embodiments, the method of manufacturing the insert of the present invention comprises mixing and reacting an electrophilic group-containing multi-arm polyethylene glycol, such as 4a20kPEG-SG, with a nucleophilic group-containing crosslinking agent, such as trilysine acetate, in a buffered solution in the presence of nepafenac particles, and allowing the mixture to gel. In certain embodiments, the molar ratio of the electrophilic groups in the PEG precursor to the nucleophilic groups in the crosslinking agent is about 1:1, but may also be in a range from about 2:1 to about 1:2.

In certain embodiments, a visualization agent as disclosed herein is included in the mixture forming the hydrogel so that the insert can be visualized once it has been administered into the canaliculus. For example, the visualization agent may be a fluorophore, such as fluorescein or a molecule comprising a fluorescein moiety, or another visualization agent as disclosed above. In certain embodiments, the visualization agent may be firmly conjugated with one or more components of the polymer network so that it remains in the insert at all times until the insert is biodegraded.

The visualization agent may for example be conjugated with either the polymer, such as the PEG, precursor, or the (polymeric or low molecular weight) crosslinking agent. In specific embodiments, the visualization agent is fluorescein and is conjugated to the trilysine acetate crosslinking agent prior to reacting the crosslinking agent with the PEG precursor. For example, in the case of fluorescein, NHS-fluorescein (N-hydroxysuccinimidyl-fluorescein) may be reacted with trilysine acetate, and completion of the formation of the trilysine-fluorescein conjugate may be monitored (e.g. by means of RP-HPLC with UV-detection). This conjugate may then be used further to crosslink the polymeric precursor(s), such as the 4a20kPEG-SG.

In certain particular embodiments, during the manufacture of an insert of the present invention a (optionally buffered) mixture/suspension of the nonsteroidal anti-inflammatory and the PEG precursor(s), such as the nepafenac and the 4a20kPEG-SG, in water is prepared. This nonsteroidal anti-inflammatory/PEG precursor mixture is then combined with a (optionally buffered) solution containing the crosslinking agent and the visualization agent conjugated thereto, such as the lysine acetate/fluorescein conjugate. The resulting combined mixture thus contains the nonsteroidal anti-inflammatory, the polymer precursor(s), the crosslinking agent, the visualization agent and (optionally) buffer. In certain embodiments, once the mixture of the electrophilic group-containing polymer precursor, the nucleophilic group-containing crosslinking agent, the nonsteroidal anti-inflammatory, such as nepafenac, optionally the visualization agent (optionally conjugated to e.g. the crosslinking agent), and optionally buffer has been prepared (i.e., after these components have been combined), the resulting mixture is cast into a suitable mold or tubing prior to complete gelling in order to provide e.g. a hydrogel strand, and ultimately the desired final shape of the hydrogel. The mixture is then allowed to gel. The resulting hydrogel is then dried.

In case the final shape of the insert is cylindrical or is essentially cylindrical, a hydrogel strand is prepared by casting the hydrogel precursor mixture comprising the nonsteroidal anti-inflammatory particles into a fine diameter tubing, such as a polyurethane (PU) tubing. Different geometries and diameters of the tubing may be used, depending on the desired final cross-sectional geometry of the hydrogel strand and thus the final insert, its initial diameter (which may still be decreased by means of stretching), and depending also on the ability of the reactive mixture to uniformly fill the tubing and to be removed from the tubing after drying. Thus, the inside of the tubing may have a round geometry or a non-round geometry, such as an oval (or other) geometry.

In certain embodiments, after the hydrogel strand has been formed and has been left to cure and to complete the gelling process within the tubing, the hydrogel strand may be longitudinally stretched in the wet or dry state as disclosed herein. The stretching may result in a dimensional change of the insert upon hydration, e.g. after it has been placed into the canaliculus. In particular embodiments, the hydrogel strand is stretched prior to (complete) drying by a stretching factor in a range of about 1 to about 3, or of about 1.5 to about 3, or of about 2.2 to about 2.8, or of about 2.5 to about 2.6. In certain embodiments, the stretching may be performed when the hydrogel strand is still in the tubing. Alternatively, the hydrogel strand may be removed from the tubing prior to being stretched. In the case dry stretching is performed in certain embodiments of the invention, the hydrogel strand is first dried and then stretched (when still inside of the tubing, or after having been removed from the tubing). When wet stretching is performed in certain embodiments of the invention, the hydrogel is stretched in a wet state (i.e., before it has dried completely) and then left to dry under tension. Optionally, heat may be applied upon stretching.

After stretching and drying the hydrogel strand may be removed from the tubing and cut into segments of a desired length, such as disclosed herein, to produce the final insert (if cut within the tubing, the cut segments are removed from the tubing after cutting). A particularly desired length for the purposes of the present invention is for example a length of equal to or less than about 3.5 mm, equal to or less than about 3.2 mm equal to or less than about 3.0 mm, or equal to or less than about 2.75 mm, such as a length in the range of about 2.0 mm to about 2.6 mm, or about 2.5 mm.

In certain embodiments, the insert in manufactured by melt extrusion or injection molding.

The method may comprise feeding the polymer composition and the active agent into an extruder; mixing the components in the extruder; extruding a strand; and cutting the strand into unit dose inserts or implants.

In certain embodiments, the polymer composition and the active agent are fed separately into the extruder. In other embodiments, the polymer composition and active agent are fed simultaneously into the extruder. In certain embodiments, the polymer composition is pre-mixed, e.g., melt blended, prior to introduction into the extruder. The mixing can be by a method using, e.g., an orbital mixer, an acoustic mixer or a v-shell blender. In certain embodiments, the polymer composition and active agent are melt blended, milled and then fed into the extruder.

In certain embodiments, the method further comprising cooling the extruded strand, e.g., prior to cutting the strand.

In certain embodiments, the method further comprises stretching the extruded strand, e.g., prior to cutting the strand.

In certain embodiments, the stretching is performed under wet or humid conditions, heated conditions, or a combination thereof. In other embodiments, the stretching is performed under dry conditions, heated conditions, or a combination thereof. In certain embodiments, strands that are stretched after crosslinking in a high humidity environment, e.g., a humidity chamber, may have shape memory or partial shape memory when placed in an aqueous environment after drying. In certain embodiments, extruded strands that are stretched or otherwise made to have smaller diameters immediately after extrusion and before crosslinking when still warm may not have shape memory.

In certain embodiments, the extruded composition is subject to a curing step, e.g., humidity exposure. When one reactant is a salt, e.g., a salt of an amine, the salt is insoluble in the dry polymer melt. In this case, curing is accomplished by exposing the dry, extruded composition to humidity and allowing the extruded composition to imbibe water from the surroundings, thus allowing the salt to solubilize and react to crosslink the precursors and form a matrix. In certain embodiments, the curing crosslinks the polymer composition.

In certain embodiments, the method further comprises drying the extruded strand after stretching the strand.

In other embodiments, any of the method steps disclosed herein can be performed simultaneously or sequentially in any order.

In certain embodiments, the method further comprises melting the polymer in the extruder at a temperature below the melting point of the active agent. The optimal temperature of the molten polymer is determined experimentally by its extrusion properties. In certain embodiments, the unmelted active agent remains unchanged through this melt extrusion process. In certain embodiments, the extrusion is performed above the melting point of the polymer and the active agent. This may result in a color change and/or change in form of the active agent, e.g., from amorphous to crystalline. The temperature can be, e.g., less than about 180°, less than about 150°, less than about 130°, less than about 120°, less than about 100°, less than about 90°, less than about 80°, less than about 70°, less than about 60°, less than about 50°. In some embodiments, the temperature is from about 50° to about 80° C. In other embodiments, the temperature is from about 50° to about 200°, about 60° to about 180° or about 80° to about 140°. An exemplary temperature is about 40° to about 90°. By virtue of certain embodiments of the present invention, the temperature is kept as low as possible to protect excipient powders and active ingredient and to optimize stability. In certain embodiments, the active agent is nepafenac and the polymer is melted in the extruder at a temperature from 57° C. to about 175° C., from about 65° C. to about 150° C. or from about 70° C. to about 90° C.

In certain embodiments, the extruded composition is dried, when in strand form or in unit doses. In certain embodiments, the drying is performed after stretching the strand. The drying can be, e.g., evaporative drying at ambient temperatures or can include heat, vacuum or a combination thereof.

In certain embodiments, the hydrogel strand is stretched by a stretch factor in the range of about 1.1 to about 10, 1.2 to about 6 or about 1.5 to about 4.

In certain embodiments, the strand is cut into segments having an average length of equal to or less than about 20 mm, 17 mm, 15 mm, 12 mm, 10 mm, 8 mm, 5 mm, 4 mm, 3 mm, 2 mm, 1 mm or 0.5 mm. In certain embodiments, the size is from about 0.5 mm to about 10 mm, about 1 mm to about 8 mm or about 1.5 mm to about 5 mm.

In certain embodiments, the active agent is suspended in the polymer composition.

In certain embodiments, the active agent is homogeneously dispersed in the polymer composition.

In certain embodiments, the extrusion process is performed without solvent (e.g., water), In certain embodiments a solvent is used in an amount of less than about 10% w/w, less than about 5% w/w or less than about 1% w/w. The solvent may be, e.g., water or an oil. An oil may result in an increased release rate for lipophilic active agents. The oil may a biocompatible vegetable oil, a synthetic oil or a mineral oil, a liquid fatty acid or triglyceride composition, or it may be a hydrophobic biodegradable liquid polymer, or combinations thereof. In certain embodiments, the oil may comprise triethyl citrate, acetyl triethyl citrate (ATEC), acetyl tributyl citrate (ATBC), α-tocopherol (vitamin E), α-tocopherol acetate; plant or vegetable oils such as sesame oil, olive oil, soybean oil, sunflower oil, coconut oil, canola oil, rapeseed oil, nut oils such as hazelnut, walnut, pecan, almond, cottonseed oil, corn oil, safflower oil, linseed oil, etc., ethyl oleate, castor oil and derivatives thereof (Cremophor®), lipids being liquid at 37° C. or lower, such as saturated or unsaturated fatty acids, monoglycerides, diglycerides, triglycerides (Myglyols®), phospholipids, glycerophospholipids, sphingolipids, sterols, prenols, polyketides, hydrophobic biodegradable liquid polymers (such as low molecular weight PLGA, PGA or PLA etc.), low melting point waxes such as plant, animal or synthetic waxes, lanolin, jojoba oil, or combinations thereof.

In certain embodiments, the content uniformity of the unit dose insert or implant is within 10%, with 5% or within 1%.

In certain embodiments, the persistence of the dosage form is from about 1 day to about 1 year, about 2 days to about 9 months or about 7 days to about 6 months after administration. This can be increased or decreased based on factors such as crosslinking.

In certain embodiments, the polymorphic form of the active agent does not change or does not substantially change. In certain embodiments, the purity of the active agent after curing is greater than 99%, greater than 99.5% or greater than 99.9% as compared to the active agent prior to extrusion. Purity is measured by chemical degradation of the active agent.

In certain embodiments, the active agent has a median (D50) particle diameter of less than about 100 μm, less than about 50 μm, less than about 25 μm, or less than about 10 μm.

In certain embodiments, the active agent has a D50 particle size of less than about 10 μm and/or a D99 particle size of less than about 50 μm, or a D90 particle size of about 5 μm or less and/or a D98 particle size of about 10 μm or less.

In certain embodiments, the polymer composition comprises polyethylene glycol, polyethylene oxide, polypropylene oxide, polyvinyl alcohol, poly (vinylpyrrolidinone), polylactic acid, polylactic-co-glycolic acid, random or block copolymers, polycaprolactone, ethylenevinyl acetate or combinations or mixtures of any of these, or one or more units of polyaminoacids, glycosaminoglycans, polysaccharides, proteins, cellulosic polymers (e.g., hydroxypropylmethylcellulose), povidone, poloxamer, acrlic polymners (e.g., polymethacrylates) or a combination thereof.

In certain embodiments, the polymer composition comprises an electrophilic group-containing multi-arm polyethylene glycol.

In certain embodiments, the polymer composition further comprises a nucleophilic group-containing crosslinking agent.

In certain embodiments, the crosslinking agent contains amine groups.

In certain embodiments, the electrophilic group-containing multi-arm-polymer precursor is 4a20kPEG-SG and the crosslinking agent is trilysine acetate.

In certain embodiments, the polymer composition further comprises a visualization agent. Ion other embodiments, the polymer composition further comprises a radiopaque agent, e.g., for x-ray or magnetic resonance imaging.

In certain embodiments, the visualization agent is a fluorophore.

In certain embodiments, the ocular insert or implant is suitable for intracanalicular, suprachoroidal, intracameral, fornix or intravitreal administration. The administration can be manually, with an insert or implant tool or device or by injection.

In certain embodiments, the extrusion process excludes water.

In certain embodiments, the present invention is directed to an ocular insert or implant prepared by a method as disclosed herein.

In certain embodiments, the present invention is directed to a method of treating an ocular disease comprising administering an ocular insert or implant as disclosed herein.

One or more of these objects of the present invention and others are solved by one or more embodiments of the invention as disclosed and claimed herein.

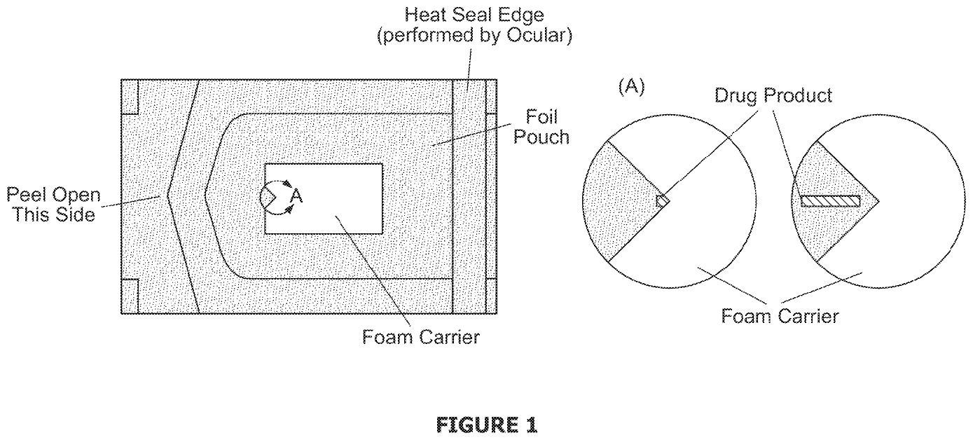

After cutting, the inserts may then be packaged into a packaging that keeps out moisture, such as a sealed foil pouch. The inserts may be fixated to a mount or support to keep them in place and to avoid damage to the insert, and also to facilitate removing the insert from the packaging and gripping/holding the insert for administration to a patient. For example, an insert of the present invention may be fixated into the opening of a foam carrier, with a portion of the insert protruding for easy removal and gripping (as illustrated in FIG. 1). The insert may be removed from the foam carrier by means of forceps and then immediately inserted into the canaliculus of a patient.

A particular embodiment of a manufacturing process according to the invention is disclosed in detail in the examples.

### Therapy

In one aspect, the present invention relates to a method of treating pain and inflammation in a patient in need thereof, the method comprising administering to the patient a sustained release biodegradable ocular (such as intracanalicular) insert as disclosed herein.

The patient to be treated in accordance with the invention may be a human or animal subject in need of pain and inflammation therapy, including acute or chronic pain and inflammation therapy. In certain embodiments, the patient may be a subject in need of acute treatment of an episodic flare of pain and inflammation.

In certain alternative embodiments, the treatment of pain and inflammation may be a long (or longer) term treatment of pain and inflammation.

In one embodiment, the present invention also relates to a sustained release biodegradable ocular (such as intracanalicular) insert as disclosed herein for use in treating pain and inflammation in a patient in need thereof.

In one embodiment, the present invention also relates to the use of a sustained release biodegradable ocular (such as intracanalicular) insert as disclosed herein for the manufacture of a medicament for treating pain and inflammation in a patient in need thereof.

Administration of the insert according to the invention is performed through the opening of the punctum into the inferior and/or superior canaliculus.

In certain embodiments, upon hydration in vivo after administration in the canaliculus the sustained release biodegradable intracanalicular insert administered to the patient increases in diameter and may decrease in length as disclosed herein.

In certain embodiments, the insert is administered to the inferior vertical canaliculus and/or the superior vertical canaliculus.

In certain embodiments, the sustained release biodegradable intracanalicular insert comprises a visualization agent such as fluorescein to enable quick and noninvasive visualization of the insert when placed inside the canaliculus. In case the visualization agent is fluorescein, the insert may be visualized by illuminating with a blue light source and using a yellow filter.

In certain embodiments, the nonsteroidal anti-inflammatory such as nepafenac or metabolite is delivered from the insert to the ocular surface through the tear film as the nonsteroidal anti-inflammatory dissolves in the tear film when released from the insert. The nonsteroidal anti-inflammatory is released primarily from the proximal end of the insert at the interface between the hydrogel and the tear fluid. The sustained nonsteroidal anti-inflammatory release rate is controlled by nonsteroidal anti-inflammatory solubility in the hydrogel matrix and the tear fluid. In certain embodiments, the nonsteroidal anti-inflammatory is nepafenac.

In certain embodiments, the insert remains in the canaliculus after complete depletion of the nonsteroidal anti-inflammatory such as nepafenac from the insert until the hydrogel has biodegraded and/or is disposed (washed out/cleared) through the nasolacrimal duct. As the hydrogel matrix of the insert is formulated to biodegrade e.g. via ester hydrolysis in the aqueous environment of the tear fluid in the canaliculus, the insert softens and liquefies over time and is cleared through the nasolacrimal duct without the need for removal. Unpleasant removal may thus be avoided. However, in case an insert should be removed e.g. because of a potential allergic reaction or other circumstances which require removal of the insert, such as an unpleasant foreign body sensation felt by a patient, or because treatment should be terminated for another reason, the insert may be expelled from the canaliculus e.g. manually.

In certain embodiments, the insert remains in the canaliculus for up to about 1 week, up to about 2 weeks, about 1 month, or up to about 2 months, or up to about 3 months, or up to about 4 months after administration.

In certain embodiments the systemic concentration of nonsteroidal anti-inflammatory such as nepafenac after administration of the insert of the present invention is very low, such as below quantifiable amounts. This significantly reduces the risk of drug-to-drug interactions or systemic toxicity, which can be beneficial e.g. in older patients who are frequently suffering from ocular diseases and are additionally taking other medications.

In certain embodiments, as the insert of the present invention is located in the canaliculus and therefore not on the surface of the eye, and only one single administration is required to provide for the release of a nonsteroidal anti-inflammatory for an extended period of time as disclosed herein, the insert does not interfere or substantially interfere with contact lenses and may therefore be particularly suitable and convenient for patients wearing contact lenses.

In certain embodiment, the patient treated with an insert of the present invention requires treatment of pain and inflammation before cataract and refractive surgery to improve outcomes/satisfaction of such surgery.

In certain embodiments, the patient treated with an insert of the present invention requires short-term treatment of signs and symptoms of pain and inflammation after cataract or refractive surgery.

In certain embodiments of the present invention, a further sustained release biodegradable intracanalicular insert is administered into the canaliculus through the ocular punctum while the first sustained release biodegradable intracanalicular insert is still retained in the canaliculus (which procedure is referred to as “insert stacking” or short “stacking”), either while the first insert still releases nonsteroidal anti-inflammatory, or after the first insert has been completely depleted of nonsteroidal anti-inflammatory, or after the first insert has been partially depleted of nonsteroidal anti-inflammatory by at least about 70%, or at least about 80%, or at least about 90% and/or the first insert releases a lower amount of nonsteroidal anti-inflammatory than initially after its administration.

In certain embodiments, insert stacking enables prolonged treatment with a nonsteroidal anti-inflammatory such as nepafenac. In certain embodiments, insert stacking thus provides for a release of a therapeutically effective amount of nonsteroidal anti-inflammatory for a total period of up to about 7 days, up to about 14 days, or up to about 28 days, or up to about 42 days, or up to about 50 days, or up to about 2 months after administration of the first insert.

### Kit

In certain embodiments, the present invention is further directed to a kit comprising one or more insert(s) as disclosed herein or manufactured in accordance with the methods as disclosed herein.

In certain specific embodiments, the kit comprises one or more sustained release biodegradable intracanalicular insert(s) as disclosed herein. In certain embodiments, the kit further comprises instructions for using the one or more sustained release biodegradable intracanalicular insert(s). The instructions for using the one or more sustained release biodegradable intracanalicular insert(s) may be in the form of an operation manual for the physician who is administering the insert(s). The kit may further comprise a package insert with product-related information.

In certain embodiments, the kit may further comprise one or more means for administration of the one or more sustained release biodegradable intracanalicular insert(s). The means for administration may be for example one or more suitable tweezer(s) or forceps, either for one time use or for repeated use. For instance, suitable forceps are blunt (non-toothed). The means for administration may also be an injection device such as a syringe or applicator system.

In certain embodiments, the kit may further comprise an ophthalmic dilator to dilate the punctum prior to the administration of the one or more sustained release biodegradable intracanalicular insert(s) and thereby facilitate insertion of the insert(s) through the punctum into the canaliculus. A dilator may also be combined/integrated with forceps or an applicator, such that e.g. one end of the device is a dilator, and the other end of the device is suitable to administer the insert. Alternatively, the kit may also contain a modified applicator that e.g. has a tapered tip that may be used for both dilation and insertion.

In certain embodiments, the one or more sustained release biodegradable intracanalicular insert(s) are individually packaged for a single administration. In certain embodiments, the one or more sustained release biodegradable intracanalicular insert(s) are individually packaged for a single administration by fixating each insert in foam carrier, which is sealed in a foil pouch. The foam carrier may have e.g. a V-notch or a circular incision with an opening at the bottom of the V-notch to hold the insert.

If two or more sustained release biodegradable intracanalicular inserts are contained in the kit, these inserts may be identical or different, and may contain identical or different doses of the nonsteroidal anti-inflammatory such as nepafenac.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Manufacturing Process of Nepafenac Intracanalicular Insert

### Drug Substance

Nepafenac is received as a GMP micronized powder.

### Nepafenac Hydrogel Insert Manufacturing

The formulation process is performed by preparing a syringe containing trilysine acetate/NHS-fluorescein/sodium phosphate dibasic and a syringe containing nepafenac/PEG-SG/sodium phosphate monobasic. The two syringes are joined together, and then mixed to create the hydrogel/nepafenac suspension, which is cast into tubing, cured, stretched, dried, and cut to length prior to packaging and sterilization.

The 4-arm polyethylene glycol is synthesized from a core molecule of pentaerythritol, which results in 4 polyethylene glycol chains per molecule having an approximate molecular weight of about 20,000 Da. The hydroxyl end groups (one per arm) of the PEG are esterified with straight chain alpha-Ω-dicarboxylic acid end groups. Each terminal carboxylic acid is esterified with an N-hydroxysuccinimidyl (NHS) leaving group for the reactive 4-arm 20K PEG SG (succinimidyl glutarate). This activated ester provides a site to react with the amino groups on the trilysine to form the hydrogel network. In a similar mechanism, a small portion of the reactive amines on the trilysine is covalently reacted with NHS-fluorescein prior to cross-linking of the hydrogel network to afford visualization via fluorescence of the hydrogel in the end product using a blue light and yellow filter.

### Preparation of Trilysine Acetate/NHS Fluorescein (TLA/FL) Syringe (Part A)

The TLA/FL syringe is a combination of trilysine acetate, NHS-fluorescein, and sodium phosphate dibasic solution. The bulk solution is prepared by mixing the ingredients at basic pH conditions for a controlled period and allowed to react for 1 to 24 hours at room temperature.

### Preparation of the Nepafenac/PEG (NPF/PEG) Syringe (Part B)

Two separate syringes are prepared and then combined to produce the nepafenac/PEG-SG syringe used to make the hydrogel. The first syringe is a suspension of the micronized nepafenac in water. The second syringe contains a solution of PEG-SG in sodium phosphate monobasic buffer. The nepafenac suspension syringe is then connected to the PEG-SG syringe with a luer connector and the contents of the syringe are passed back until mixed. The suspension is then transferred into one syringe to form the nepafenac/PEG-SG syringe.

### Mixing and Casting of the Hydrogel

To form the hydrogel/nepafenac suspension, the TLA/FL syringe (Part A) and NPF/PEG-SG syringe (Part B) are connected by a luer connector. The contents of the syringe are passed back until mixed creating the reactive suspension of hydrogel/nepafenac which is transferred into a single syringe. The hydrogel precursors/nepafenac suspension syringe is then connected to the barb fitting on the tubing and the suspension is injected into the tubing. Typical tubing diameters utilized are 2.0 to 2.2 mm but may be adjusted accordingly based on needs to generate insets with different dried and/or hydrated diameters. Once the tubing is full, the tubing is removed from the syringe and barb fittings on the tube are capped. The remaining pieces of tubing are then filled in the same manner. At this point, a filled tube containing the hydrogel/nepafenac suspension is referred to as a casted strand. The formulation and casting process are repeated as necessary to prepare the desired number of strands per batch. The casted strands are stored for approximately 2 to 24 hours to allow the gel to fully react (cure).

### Stretching and Drying

An incubator set to approximately 32.0° C. with a nitrogen air flow is used for drying. Once the cure time has elapsed, the casted strands are placed in the stretching fixture and secured in place with dynamic clamps. The casted strands are stretched on the stretching fixture to approximately 2.5× the original tubing length. The stretching fixtures are then moved to the incubator for approximately 3 days (or until the strands are fully dry) prior to removal and cutting.

### Cutting and Inspection

After the drying time has elapsed, the stretching fixtures with the dried strands are removed from the incubator. The tubing containing the casted strand is cut from the stretching fixture. The dried strand is removed from the tubing. The strands are processed through the cutter and cut into approximately 3.0 mm lengths. The cut inserts are stored in vials under nitrogen until packaging.

### Packaging and Container Closure System

The insert is placed in a foam carrier, sealed within a desiccant impregnated heat sealable low vapor-transmission aluminum-LDPE laminate foil pouch (Amcor Dessiflex™) under a nitrogen environment. The pouched inserts are terminally sterilized via gamma irradiation. The packaged product is then stored under refrigerated conditions between 2° C. and 8° C. A schematic of the packaging configuration is shown in FIG. 1.

### Example 2—Nepafenac Inserts Used in Beagle Pharmacokinetic Study

The insert used in a completed low dose pharmacokinetic study was formulated to contain approximately 0.4 mg of nepafenac having a dried diameter of 0.5 mm and length of 3.0 mm. Upon 24 hour hydration in PBS, pH 7.4 at 37° C. the inserts hydrated in diameter to 1.5 mm and shortened in length to 2.7 mm. The insert used in an in progress high dose pharmacokinetic study was formulated to contain approximately 0.6 mg of nepafenac having a dried diameter of 0.6 mm and length of 3.0 mm. Upon 24 hour hydration in PBS, pH 7.4 at 37° C. the inserts hydrated in diameter to 1.8 mm and shortened in length to 2.8 mm. Critical variables of the two lots used in the PK studies covering the formulation, casting/stretching variables, packaging, and sterilization, along with dimensional and mass outputs are listed in the table below.

| Implant type | | | | | |
|---|---|---|---|---|---|
| Formulation | Drug Type | Nepafenac(low dose) | | Nepafenac(high dose) | |
| (% dry | Drug | 53.4% | | 64.8% | |
| basis w/w) | Theoretical Dose | 400 | ug | 600 | ug |
| | (as formulated - not measured) | | | | |
| | Measured dose | 411 | ug | 596 | ug |
| | PEG Hydrogel | | | | |
| | 4a20K PEG-SG | 41.1% | | 31.2% | |
| | Trilysine Acetate | 1.2% | | 0.9% | |
| | NHS-Fluorescein | 0.4% | | 0.1% | |
| | Sodium phosphate (total) | 4.0% | | 3.0% | |
| | Sodium phosphate monobasic | 1.1% | | 0.8% | |
| | Sodium phosphate dibasic | 2.9% | | 2.2% | |
| Formulation | Drug | 9.8% | | 14.8% | |
| (% wet | PEG Hydrogel | | | | |
| basis w/w) | 4a20K PEG-SG | 7.6% | | 7.15% | |
| | Trilysine Acetate | 0.2% | | 0.2% | |
| | NHS-Fluorescein | 0.02% | | 0.02% | |
| | Sodium phosphate (total) | 0.7% | | 0.7% | |
| | Sodium phosphate monobasic | 0.2% | | 0.2% | |
| | Sodium phosphate dibasic | 0.5% | | 0.5% | |
| | WFI | 81.6% | | 77.1% | |
| Configuration | Drug per final dry length | 133.3 | ug/mm | 200 | ug/mm |
| | (Theoretical Dose/Cut Length) | | | | |
| | Approximate Implant Mass | 749 | | 926 | |
| | (dose μg/API %) | | | | |
| | Wet Stretch Factor | 2.5 | | 2.5 | |
| | Packaging | Foil Pouches | | Foil Pouches | |
| | Sterilization Type | Gamma | | Gamma | |
| | Site Storage | Refrigerated | | Refrigerated | |
| Dimensions | Dried | | | | |
| | Diameter | 0.50 ± 0.01 | mm | 0.58 ± 0.02 | mm |
| | Length | 2.99 ± 0.02 | mm | 3.03 ± 0.04 | mm |
| | Volume | 0.59 ± 0.02 | mm³ | 0.80 ± 0.04 | mm³ |
| | Implant Mass | 0.69 | mg | 0.96 | mg |
| | Drug per volume | 678.0 | | 750.0 | |
| | (μg/mm³) | | | | |
| | Hydrated | | | | |
| | Diameter | 1.54 ± 0.03 | mm | 1.825 ± 0.02 | mm |

| Length | 2.67 ± 0.07 | 2.79 ± 0.07 |
|---|---|---|
| Tubing ID | 2.0 | 2.0 |
| (mm) | | |

The low dose nepafenac inserts were evaluated for drug release in a 28 day pharmacokinetic study in beagle dogs to assess the drug release profile of both nepafenac and the active metabolite amfenac in tear fluid and aqueous humor. A hydrogel nepafenac intracanalicular insert was placed bilaterally into the inferior canaliculus of 20 beagle dogs on day 0. After presence of the insert in the canaliculus was confirmed visually with a blue light source and aided with a yellow filter, tear fluid was collected from eyes with pre-cut 10 mm Schirmer test strips at 2 hours, and 1, 3, 7, 10, 14, 17, 21, 24, and 28 days post-insertion. Aqueous humor was collected from n=6 eyes on days 7, 14, 21 and 28. Tear fluid and AH samples were analyzed for nepafenac and amfenac by liquid chromatography tandem mass spectrometry (LC-MS/MS). Ten unused inserts returned from the study were extracted for nepafenac and analyzed by LC-MS/MS and results demonstrated a good assay consistency of 413±15 mcg with a range from 393 to 436 mcg.

The average and standard deviation concentrations of nepafenac, amfenac and total concentrations (both analytes) in tear fluid samples post-insertion are presented in the table below. The drug release profile demonstrated sustained release for approximately 10 days followed by a tapering over and almost full clearance from the tear fluid measurements by days 14 and 17.

The average and standard deviation of amfenac in aqueous humor samples at 7, 14, 21 and 28 days is presented in the table below. Results demonstrated elevated levels of amfenac at 7 days in the AH and minimal levels at 14 days. All results for amfenac were below the limit of quantitation at 21 and 28 days consistent with the tear fluid levels. All results for nepafenac were below the limit of quantitation at all time points consistent with the conversion of nepafenac to the active metabolite amfenac as it passes through the cornea. levels.

Average and Standard Deviation of Nepafenac,
Amfenac and Total Concentrations of in Beagle
Tear Fluid Over the OT-2101 PK Study Duration

| | | | | | | Nepafenac and | |
|---|---|---|---|---|---|---|---|
| | | Nepafenac | | Amfenac | | Amfenac | |
| Time | | Average | Std Dev | Average | Std Dev | Average | Std Dev |
| (days) | N | (ng/mL) | (ng/mL) | (ng/mL) | (ng/mL) | (ng/mL) | (ng/mL) |
| 0.1 | 9 | 671 | 261 | 153 | 78 | 824 | 331 |
| 1 | 10 | 594 | 262 | 79 | 49 | 673 | 297 |
| 3 | 10 | 312 | 165 | 89 | 140 | 401 | 170 |
| 7 | 10 | 211 | 84 | 124 | 210 | 335 | 236 |
| 10 | 10 | 138 | 116 | 39 | 44 | 177 | 153 |
| 14 | 14 | 5 | 7 | 4 | 8 | 9 | 14 |
| 17 | 10 | 1 | 3 | 0 | 0 | 1 | 3 |
| 21 | 10 | 0 | 0 | 0 | 0 | 0 | 0 |
| 24 | 10 | 0 | 0 | 0 | 0 | 0 | 0 |
| 28 | 7 | 0 | 0 | 0 | 0 | 0 | 0 |

Average and Standard Deviation of Amfenac in Beagle
Aqueous Humor Over the OT-2101 PK Study Duration

| Time | Average | Std Dev |
|---|---|---|
| (days) | (ng/mL) | (ng/mL) |
| 7 | 18.3 | 8.4 |
| 14 | 1.3 | 1.5 |
| 21 | 0.0 | 0 |
| 28 | 0.0 | 0 |

The half maximal inhibitory concentration (IC50) of amfenac for the cyclooxygenase-2 (COX-2) enzyme responsible for inflammation and pain is approximately 0.45 ng/ml (Walters et al., 2007). The insert at 7 days delivered amfenac into the aqueous humor in beagle dogs at concentrations approximately 40.6-fold the IC50 indicating the potential to inhibit pain and inflammation through the COX-2 pathway.

The high dose insert containing 0.6 mg of nepafenac is currently being evaluated a study in beagle dogs. This higher dose containing insert is expected to provide increased levels of amfenac into the aqueous humor for a longer duration of time.

### Example 3 Nepafenac Micronized API Characterization of Size Distribution With Laser Diffraction Representative Size Distribution

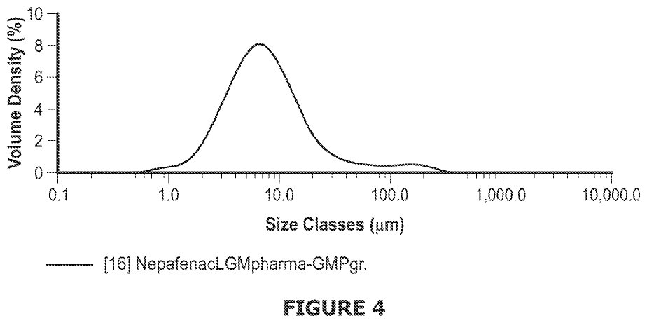

The size distribution of the nepafenac active pharmaceutical ingredient was obtained by laser diffraction and the median size distribution value of the micronized API had a D₅₀: 7.03 μm and ninety percent of the particles had diameters below D₉₀: 23.4 μm. The results are below and in FIG. 4.

| | Record Number | Sample Name | Dx (10) (μm) | Dx (50) (μm) | Dx (90) (μm) |
|---|---|---|---|---|---|
| | 16 | Nepafenac | 2.76 | 7.03 | 23.4 |
| Mean | | | 2.76 | 7.03 | 23.4 |

### Example 4—In Vitro Release Profile of Nepafenac Inserts

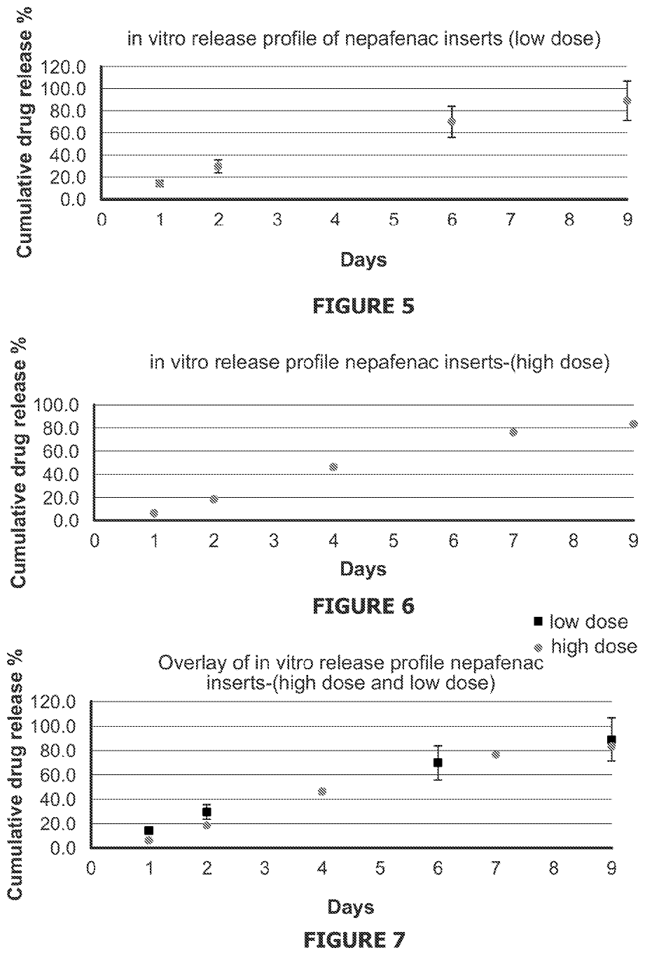

In vitro release profile of the test articles was evaluated in 1×PBS (pH 7.4) at 37 C. N=5 Results indicate that low dose nepafenac test articles had an average nepafenac release rate of at day 1: 14.1% and day 9: 88.9%. Summarized results are shown the table below and FIG. 5.

| | | |
|---|---|---|
| In Vitro | Percent drug release of nepafenac | Day 1: 14.1% |
| Drug | from low dose test articles | Day 2: 29.6% |
| Release | | Day 6: 69.8% |
| profile | | Day 9: 88.9% |

Results indicate that high dose nepafenac test articles had an average nepafenac release rate of at day 1: 14.1% and day 9: 88.9%. Summarized results are shown in the table below and FIG. 6.

| | | |
|---|---|---|
| In Vitro | Percent drug release of nepafenac | Day 1: 6.3% |
| Drug | from high dose test articles | Day 2: 18.5% |
| Release | | Day 4: 46.3% |
| profile | | Day 7: 76.4% |
| | | Day 9: 83.4% |

An overlay of in vitro release profile nepafenac inserts of high dose and low dose test articles is shown in FIG. 7.

### Example 5—High Dose PK Study Nepafenac and Amfenac Concentrations in Beagle Aquoeus Humor

The average and standard deviation of nepafenac and amfenac in aqueous humor samples at 0.125, 1, 7, 14, 21,28 and 42 days is presented in the table below. Results demonstrated elevated levels of amfenac at 1 days in the AH and minimal levels at 14 days. All results for amfenac were below the limit of quantitation at 21 and 42 day. All results for nepafenac in aqueous humor were below the limit of quantitation at all time points consistent with the conversion of nepafenac to the active metabolite amfenac as it passes through the cornea.

### Average and Standard Deviation of Nepafenac and Amfenac Concentrations in Beagle Aquoeus Humor Over the Study Duration

| | |
|---|---|
| Nepafenac | Amfenac |

| Time | | Average | Std Dev | Average | Std Dev |
|---|---|---|---|---|---|
| (days) | N | (ng/mL) | (ng/mL) | (ng/mL) | (ng/mL) |
| 0.125 | 6 | 0 | 0 | 0.8 | 1.4 |
| 1 | 6 | 0 | 0 | 65.6 | 46.3 |
| 7 | 6 | 0 | 0 | 14.7 | 10.6 |
| 14 | 6 | 0 | 0 | 8.6 | 3.5 |
| 21 | 6 | 0 | 0 | 0 | 0 |
| 28 | 6 | 0 | 0 | 0 | 0 |
| 42 | 6 | 0 | 0 | 0 | 0 |

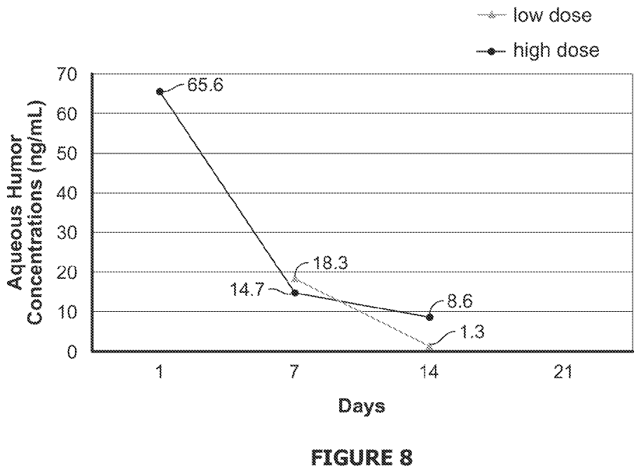

An overlay of Amfenac Concentrations in Beagle Aquoeus humor for high and low dose nepafenac inserts is shown in FIG. 8.

### A summary of Examples is Presented Below

### Purpose

Topical nonsteroidal anti-inflammatory drugs (NSAIDs) are often prescribed following ophthalmic surgery to reduce ocular pain and modulate inflammation. Sustained-release drug delivery of NSAIDs may overcome some limitations of topical therapy such as patient self-dosing and nonadherence. Here we evaluate the pharmacokinetics of 0.4 mg nepafenac delivered from a biodegradable hydrogel intracanalicular insert in a canine model.

### Methods

A hydrogel nepafenac (0.4 mg) intracanalicular insert was place bilaterally into the inferior canaliculus of 20 beagle dogs on Day 0. After presence of the insert in the canaliculus was confirmed visually, tear fluid was collected from n=10 eyes with pre-cut 10 mm Schirmer test strips at 2 hours, and 1, 3, 7, 10, 14, 17, 21, 24 and 28 days post-insertion. Aqueous humor (0.1 mL, n=6 eyes) was collected 7, 14, 21, and 28 days post-insertion. Tear fluid and aqueous humor samples were analyzed for nepafenac (prodrug) and amfenac (active metabolite) by liquid chromatography tandem mass spectrometry. Any remaining inserts were removed from the animals at Day 28 and analyzed for drug content.

### Results

Maximum mean concentration of nepafenac (671 ng/ml) and amfenac (153 ng/ml) in the tear fluid was measured at 2 hours post-insertion indicating rapid dissolution of drug from the insert soon after placement. Mean nepafenac and amfenac levels in tear fluid samples over time showed drug levels gradually tapered over time. Nepafenac and amfenac levels were cleared from the tear fluid by 10-14 days. Both the prodrug and active metabolite were found in the tear fluid in an 85:15 ratio, however, only amfenac was detected in aqueous humor samples. Amfenac concentration in the aqueous humor was highest on Day 7 at 18.3 ng/ml and decreased to 2.6 ng/ml at Day 14. Levels of amfenac in the AH were 41-fold above the half maximal inhibitory concentration of cyclooxygenase-2 for amfenac (COX-2 IC₅₀=0.45 ng/ml) indicating potential therapeutic benefit at these concentrations.

### CONCLUSIONS

A hydrogel-based intracanalicular insert with 0.4 mg nepafenac quickly released drug at therapeutic levels to the ocular surface and continued for 10-14 days.

1. A sustained release biodegradable ocular insert comprising a hydrogel and from about 0.1 to about 1.2 mg nepafenac or a pharmaceutically acceptable salt thereof, wherein the nepafenac is dispersed within the hydrogel.

2. The sustained release biodegradable ocular insert of claim 1, wherein the insert contains from about 0.1 to about 1.0 mg nepafenac.

3. The sustained release biodegradable insert of claim 1, wherein the insert provides for a release of a therapeutically effective amount nepafenac for a period of up to about 2 weeks or 1 month after administration.

4. (canceled)

5. The sustained release biodegradable ocular insert of claim 1, wherein the insert is an intracanalicular insert.

6. (canceled)

7. (canceled)

8. (canceled)

9. (canceled)

10. (canceled)

11. (canceled)

12. (canceled)

13. (canceled)

14. The sustained release biodegradable ocular insert of claim 1, wherein the insert is cylindrical or essentially cylindrical and in its dry state has a length of less than about 3.5 mm, less than about 3.2 mm or less than about 3.0 mm.

15. (canceled)

16. (canceled)

17. The sustained release biodegradable ocular insert of claim 1, wherein the insert is cylindrical or essentially cylindrical and upon hydration (after 24 hours in phosphate-buffered saline at a pH of 7.2 at 37° C.) the diameter of the insert is increased and the length of the insert is decreased.

18. The sustained release biodegradable ocular insert of claim 17, wherein the ratio of the diameter of the insert in the hydrated state to the diameter of the insert in the dry state is in the range of about 1.5 to about 4.

19. (canceled)

20. (canceled)

21. (canceled)

22. The sustained release biodegradable ocular insert of claim 1, wherein the insert is cylindrical or essentially cylindrical and in its hydrated state (after 24 hours in phosphate-buffered saline at a pH of 7.2 at 37° C.) has a ratio of length to diameter of greater than 1 or greater than 1.2 or greater than about 1.4 or greater than about 1.7.

23. The sustained release biodegradable ocular insert of claim 1, wherein the insert is cylindrical or essentially cylindrical and in its hydrated state has a diameter in the range of about 1.2 mm to about 2.2 mm and a length in the range of about 2.0 mm to about 3.0 mm.

24. The sustained release biodegradable ocular insert of claim 1, having a total weight in the range of about 50 to about 1200 μg.

25. (canceled)

26. The sustained release biodegradable ocular insert of claim 1, wherein the insert provides for the release of a therapeutically effective amount of nepafenac for a period of about 12 hours or longer after administration.

27. (canceled)

28. (canceled)

29. The sustained release biodegradable ocular insert of claim 1, wherein the insert provides for the release of nepafenac to provide clinically effective drug levels of amfenac for a period of up to about 5 days after administration.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. The sustained release biodegradable ocular insert of claim 1, wherein the nepafenac has a d90 particle size of equal to or less than about 10 or 5 μm and/or a d50 particle size of equal to or less than about 6 or 3 μm and/or d10 particle size of equal to or less than about 2 or 1 μm.

47. (canceled)

48. (canceled)

49. The sustained release biodegradable ocular insert of claim 1, wherein the insert is free or substantially free of antimicrobial preservatives.

50. A method of manufacturing the sustained release biodegradable ocular insert according to claim 1, the method comprising the steps of forming a hydrogel comprising a polymer network and nepafenac particles dispersed within the hydrogel, shaping the hydrogel and drying the hydrogel.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method of treating pain and inflammation in a patient in need thereof, the method comprising administering to the patient a sustained release biodegradable ocular insert according to claim 1.

64. The method of claim 63, wherein the treatment is for one or more of pain and inflammation after cataract surgery.

65. A method of treating pain and inflammation in a patient in need thereof, the method comprising administering intracanalicularly to the patient a sustained release biodegradable ocular insert comprising a hydrogel and nepafenac, wherein nepafenac particles are dispersed within the hydrogel.

66. The method of claim 65, wherein the treatment period is up to about 14 days or 1 month.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. A kit comprising one or more sustained release biodegradable ocular insert(s) of claim 1 and instructions for using the insert(s), wherein the insert(s) is/are individually packaged for a single administration.

81. (canceled)

82. (canceled)

83. (canceled)

84. (canceled)

85. (canceled)

86. (canceled)

87. (canceled)
